(19) United States
(12) Patent Application Publication
(10) Pub. No.: US 2025/0215514 A1
(43) Pub. Date: Jul. 3, 2025

(54) LOW-DENSITY LIQUID-PHASE CHIP FOR GOSSYPIUM HIRSUTUM L. BASED ON TARGETED CAPTURE SEQUENCING AND USE THEREOF

(71) Applicant: ZHEJIANG UNIVERSITY, Hangzhou (CN)

(72) Inventors: Zhanfeng SI, Hangzhou (CN); Yan HU, Hangzhou (CN); Tianzhen ZHANG, Hangzhou (CN); Lei FANG, Hangzhou (CN); Zegang HAN, Hangzhou (CN)

(21) Appl. No.: 19/023,226

(22) Filed: Jan. 15, 2025

Related U.S. Application Data
(63) Continuation of application No. PCT/CN2024/099474, filed on Jun. 17, 2024.

(30) Foreign Application Priority Data
Jun. 27, 2023 (CN) 202310765880.0

(51) Int. Cl. C12Q 1/6895; C12Q 1/6874

(52) U.S. Cl. CPC C12Q 1/6895; C12Q 1/6874; C12Q 2600/13

(57) ABSTRACT

A low-density liquid-phase chip for Gossypium hirsutum L. based on targeted capture sequencing and use thereof provided. The low-density chip includes 908 SNP loci, where among the 908 SNP loci, 329 loci are significantly related to important agronomic traits, such as fiber quality, yield, and disease resistance of Gossypium hirsutum L., 14 loci are common plant transgenic detection loci, and 565 loci are other loci. This chip is adapted for use of detection of transgenic components in Gossypium hirsutum L. varieties, resource evaluation and lineage identification, seed purity identification, and genetic improvement of major agronomic traits of Gossypium hirsutum L. varieties.

### CROSS-REFERENCE TO RELATED APPLICATIONS

The present application is a National Stage of International Application No. PCT/CN2024/099474 filed on Jun. 17, 2024, which claims a priority to Chinese Patent Application No. 2023107658800, filed on Jun. 27, 2023, both of which are hereby incorporated by reference in their entireties.

### REFERENCE TO SEQUENCE LISTING

The present application is being filed along with a Sequence Listing in electronic format. The Sequence Listing is provided as a file entitled with “DF242868US-Sequence Listing ST.26”, and created on Jan. 9, 2025, which is approximately 20.3 KB in size. The information in the electronic format of the Sequence Listing is incorporated herein by reference in its entirety.

### TECHNICAL FIELD

The present disclosure relates to the fields of genetics, molecular biology, bioinformatics and cotton molecular breeding, in particular to a Low-density liquid-phase chip for Gossypium hirsutum L. based on targeted capture sequencing and use thereof.

### BACKGROUND

Single Nucleotide Polymorphism (SNP) refers to the variation of a single nucleotide in a genome, which is a variation form formed by substitution, transversion, insertion or deletion of a single nucleotide pair. As the third generation genetic marker, single nucleotide polymorphism is an ideal genotyping target because of a large number of SNPs, dense distribution and easy detection. In view of the innovation of molecular marker technology, single nucleotide polymorphism (SNP) based on DNA sequence variation is close to the ultimate standard for mutation detection at the molecular level. It is expected that the development of molecular detection technology and platform in the next few years will be promoted and improved on this basis. Therefore, efficient and low-cost SNP genotyping technology has become the best choice for developing sharing technology and platform.

Genotyping by target sequencing (GBTS) technology based on SNP is to select specific target loci from genomic DNAs for sequencing and genotyping. This targeted or fixed simplified genome sequencing greatly reduces the amount of DNA sequencing, simplifies biological information analysis and data processing, and improves the adaptability to various genotyping platforms. Moreover, this technique is adapted for the detection of all marker loci, including known loci (such as cloned genes), functionally unknown loci (candidate genes) and neutral loci. The flexibility of the number of systematic markers provides a huge elastic space for various applications, which is basically adapted for all scenarios requiring different numbers of markers, including e.g., marker-assisted major gene selection, backcross breeding and its background selection, polygenic polymerization breeding, seed purity detection, transgenic component identification and the like. Therefore, GBTS will be the first choice for genotyping for a long time to come.

Cotton SNP chips, such as Illumina 80K and ZJU40K, that have been developed have been widely used by breeding enterprises and scientific research units for genotyping, genome selection and genetic diversity analysis of germplasm resources. The SNP density of these chips is generally high, and the Illumina 80K solid-phase chip still has the disadvantage of being unable to be customized. The design of chip loci is generally based on the evenly distributed loci in the whole genome, without considering the correlation with traits, therefore the typing cost is high and the purposiveness of selection is not strong. Traditional breeding has a long cycle and unpredictable phenotypic variation, which mainly depends on the experience of breeders. Other excellent traits may be easily lost when improving the breeding target traits. Molecular marker-assisted breeding is to use the characteristics of close linkage between molecular markers and genes that determine target traits. By detecting molecular markers, the target genes and loci are detected to achieve the purpose of selecting target traits which has the advantages of rapidness, accuracy and no interference from environmental conditions, with its key limiting factor being the accurate identification of loci associated with traits.

To sum up, it is urgent to develop a low-density liquid-phase SNP chip for Gossypium hirsutum L. genotyping, transgenic detection, germplasm resource evaluation and molecular marker-assisted selection based on the loci significantly correlated with Gossypium hirsutum L. fiber quality, yield, disease resistance and other important agronomic traits excavated by genome sequencing and genome-wide association study of Gossypium hirsutum L.

### SUMMARY

In view of the shortcomings of the prior art, it is an object of the present disclosure to find and screen 894 SNP loci, and develop a liquid-phase gene chip by using the 894 SNP loci and 14 major transgenes of Gossypium hirsutum L. for detecting transgenic components and genotyping of Gossypium hirsutum L.

The technical solution adopted by the present disclosure is as follows:
the present disclosure relates to a Low-density liquid-phase chip for Gossypium hirsutum L. based on targeted capture sequencing. A whole genome low-density SNP chip of Gossypium hirsutum L. consists of probes for detecting 894 SNP loci and 14 major transgenes;
the 14 major transgenes are specifically: A1Bt, A2CPTI, A3CP4-epsps, A4CP4-epsps, A5bar, A6pat, A7CdP450, A8cp4-epsps, B.1CaMV35S, B.2FMV35S, B.3NOS, B.4NOS, B.5CaMV35S and NPTII; the 894 SNP loci are shown in Table 1.

Further, the probes for detecting the 14 major transgenes are shown as SEQ ID NOs. 1-14.

The gene chip of that present disclosure is a liquid-phase chi, in which probes are designed based on the liquid-phase probe capture technology to synthesize a kit containing information of 908 loci based on, and can be used for detecting whether there are 14 common transgenic components in Gossypium hirsutum L., Gossypium hirsutum L. resource evaluation, lineage identification, seed purity identification and genetic improvement of major agronomic traits of Gossypium hirsutum L. varieties.

Further, the major agronomic traits comprise fiber quality trait, yield trait and disease resistance.

The liquid-phase chip technology system used in the present disclosure is a GenoBaits technology system. The working principle is that the probes designed based on the target SNP markers carries out complementary combination and sequencing of the target sequences with the DNA of the test sample, so as to achieve the purpose of genotyping of the target SNP marker of the test sample. In the present disclosure, all probes that can accurately detect 894 SNP loci and 14 major transgenes can achieve the same technical effect. The use of the chip can greatly improve the accuracy of genotyping, improve the detection level, reduce the detection cost, and provide important technical support for cotton molecular breeding.

### BRIEF DESCRIPTION OF DRAWINGS

FIG. 1 is a statistical diagram of SNP marker chromosome distribution; statistical results of the number of SNP markers on different chromosomes: a. the abscissa is the chromosome number; B. the left ordinate indicates the number of loci and segments; c. the right ordinate indicates the chromosome length (Mb);

FIG. 2 shows the distribution of SNP loci on chromosomes.

### DESCRIPTION OF EMBODIMENTS

The present disclosure will be further described in detail with reference to specific embodiments.

### Example 1: Design and Preparation of the Low-Density Liquid-Phase Chip for Gossypium hirsutum L. Based on Targeted Capture Sequencing

The specific method is as follows:

1. Based on VCF files obtained by resequencing three populations (Fang et al. Genomic analyses in cotton identify signatures of selection and loci associated with fiber quality and yield traits. Nature genetics, 2017, 49 (7): 1089; Liu et al. Association mapping of seed oil and protein contents in upland cotton. Euphytica, 2015, 205 (2): 637-645), and taking the cotton genome TM-1_V2.1 (Hu et al. Gossypium barbadense and Gossypium hirsutum genomes provide insights into the origin and evolution of allotetraploid cotton. Nature genetics, 2019, 51 (4): 739-748.) as a reference, 601 SNP loci (background loci) were screened according to the principles of MAF>0.35, heterozygosity rate<15%, NA ratio<10% and uniform chromosome distribution for the SNP loci in the populations.

2. According to the GWAS association results of major agronomic traits (Fang et al. Genomic analyses in cotton identify signatures of selection and loci associated with fiber quality and yield traits. Nature genetics, 2017, 49 (7): 1089; Liu et al. Association mapping of seed oil and protein contents in upland cotton. Euphytica, 2015, 205 (2): 637-645; Li et al. Genomic Insights into the Genetic Basis of Cotton Breeding in China, Molecular Plant 16, 662-677), 634 SNP loci (foreground loci) significantly related to agronomic traits and disease resistance were selected.

3. A total of 1235 target segments of Gossypium hirsutum L. genomes were obtained, and 1148 candidate target segments were obtained after screening by detecting whether the positions and locus genotypes of candidate SNPs were consistent with the reference genome TM-1V2.1 (cotton.zju.edu.cn) of Gossypium hirsutum L.

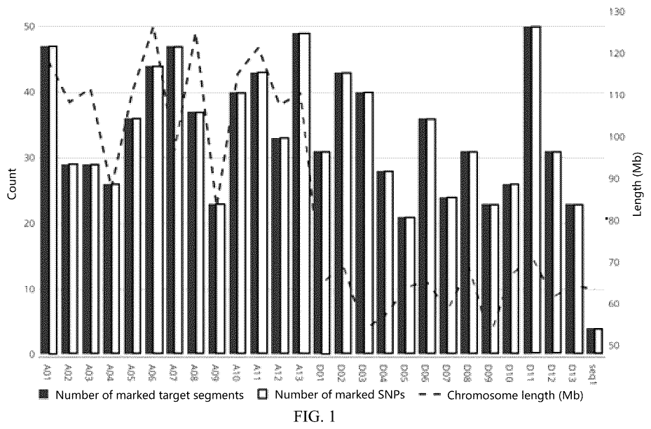

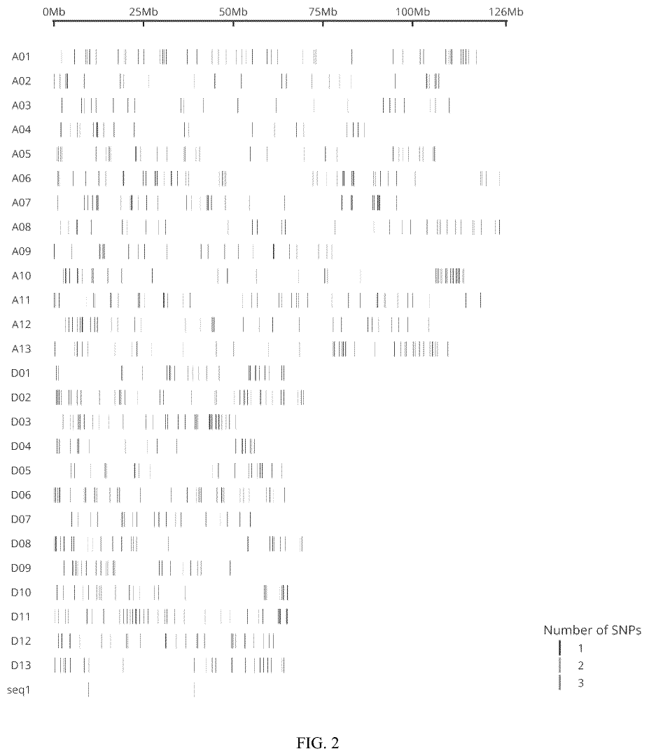

4. According to 1148 candidate target segments, probe design was carried out. The design principles were as follows: all probes with a length of 100-120 bp in the coverable area were selected; the GC content of the probe in the coverable target region was calculated; the number of homology regions of the probe was calculated, where the three situations, namely, a. 40 bp above being exactly the same, b. the similarity being 85% and the length being 80 bp and c. the similarity being 95% and the length being 70 bp, were regarded as a homology region. The probe selection principles were as follows: selecting probes with a GC content between 30% and 70%; the number of homology regions being less than or equal to 5; the selected probe region not containing SSR and N regions. According to the above design and selection principles, a total of 1795 probes were designed and selected, which could detect 894 target segments. The information of core SNP loci corresponding to 894 target segments is shown in Table 1, and the statistics of SNP marker chromosome distribution are shown in FIG. 1 and FIG. 2.

5. According to the 14 common detection sequences of cotton transgenic detection published by the Ministry of Agriculture, 14 detection probes were designed. The 14 detection probes provided in this example are shown as SEQ ID NOs. 1-14, which respectively detected A1Bt, A2CPTI, A3CP4-epsps, A4CP4-epsps, A5bar, A6pat, A7CdP450, A8cp4-epsps, B.1CaMV35S, B.2FMV35S, B.3NOS, B.4NOS, B.5CaMV35S and NPTII genes; the high-throughput sequencing sum analysis for the amplified product of the primer pair combination can be carried out for one time to complete the detection of transgenic components in the sample. Among them, the probes (SEQ ID NO. 3 and SEQ ID NO. 12) for detecting the A3CP4-epsps gene and B.4NOS gene are shown in the table; where I is hypoxanthine.

| | Sequence | | Gene |
|---|---|---|---|
| | length | Sequence information | name |
| SEQ ID NO. | 333 | ACGGTGACCG TCTTCCCGTT ACCITGCGCG GGCCGAAGAC | A3CP4- |
| 3 | | GCCGACGCCG ATCACCTACC GCGTGCCGAT GGCCTCCGCA | epsps |
| | | CAGGTGAAGT CCGCCGTGCT GCTCGCCGGC CTCAACACGC | |
| | | CCGGCATCAC GACGGTCATC GAGCCGATCA TGACGCGCGA | |
| | | TCATACGGAAAAGATGCTGC AGGGCTTTGG CGCCAACCTT | |
| | | ACCGTCGAGA CGGATGCGGA CGGCGTGCGC ACCATCCGCC | |
| | | TGGAAGGCCG CGGCAAGCTC ACCGGCCAAG TCATCGACGT | |
| | | GCCGGGCGAC CCGTCCTCGA CGGCCTTCCC GCTGGTTGCG | |
| | | GCCCTGCTTG TTC | |
| SEQ ID NO. | 217 | ATCGTTCAAA CATTTGGCAA TAAAGTTTCT TAAGATTGAA | B.4NOS |
| 12 | | TCCTGTTGCC GGTCTTGCGA TGATTATCAT ATTAATTICTG | |
| | | TTGAATTACG TTAAGCATGT AATAATTAAC ATIGTAATCCA | |
| | | TGACGTTATT TATGAGATGG GTTTTTATGA TTAGAGTCCC | |
| | | GCAATTATAC ATTTAATACG CGATAGAAAA CAATATAG | |
| | | CGCGCAAACT AGGATAA | |

6. The probes for detecting 894 SNP loci and 14 major transgenes were composed into a Low-density liquid-phase chip for Gossypium hirsutum L. based on targeted capture sequencing; wherein, 329 loci were significantly related to important agronomic traits such as fiber quality, yield and disease resistance of Gossypium hirsutum L., 14 loci were common plant transgenic detection loci, and 565 were other loci.

In this example, the capture probes synthesized by MOLBREEDING Biotechnology Co., Ltd. were used to construct the chip, which was based on the targeted genotyping (GBTS) technology of liquid phase probe hybridization: GenoBaits. The working principle of GenoBaits is based on the complementary combination of target probes and target sequences for fixed-point capture. Firstly, the gDNA library of the materials to be tested was constructed. At the same time, a probe covering the target SNP was designed at each locus to be tested according to the principle of DNA complementarity, and the target probe was labeled with a Biotin label. Then, in a liquid state, the target probe labeled with Biotin hybridized with the target region of the genome to form a double strand. Subsequently, the target probe with the Biotin label was subjected to molecular adsorption by magnetic beads coated with streptavidin, so as to capture the target that hybridized with the probe. Finally, elution, target amplification, library building and sequencing were carried out on the captured target sequence, and finally the genotype of the target SNP was obtained.

The 894 SNP loci studied by the inventor are involved by Gossypium hirsutum L. itself, while are selected with full consideration of the distribution of these SNPs on chromosomes, and of the detection efficiency of the probes designed based on SNP markers in the population. Among them, 329 SNP loci are significantly associated with important agronomic traits, and 565 SNP loci are background loci. Therefore, the probes designed adopting these SNP markers can directly select target traits in breeding, as well as detecting the recovery rate of genetic background. For example, breeding target loci improve fiber quality, thus individuals with excellent haplotypes and high background recovery rate can be selected from the population based on fiber associated SNP markers, thereby enhancing the targeting of breeding, and accelerating the breeding process. The chip also includes probes for 14 major transgenes, allowing for direct detection of transgenes during the breeding process. At present, the transgenic detection is generally targeted at a single gene/promoter sequence, so detecting 14 target fragments requires 14 PCR reactions, making the detection method cumbersome. The chip developed by the inventor can detect 14 major transgenic sequences while detecting 894 SNP haplotypes, improving detection efficiency.

TABLE 1
Information of 894 core SNP loci

| | | | | | Whether |
|---|---|---|---|---|---|
| | | | | | it is a |
| SNP | | Genome | | | functional |
| number | Chromosome | position | Ref | Alt | locus |
| 001 | A01 | 110747518 | C | T | Yes |
| 002 | A03 | 11727109 | T | C | Yes |
| 003 | A03 | 20665441 | A | G | Yes |
| 004 | A03 | 2166240 | A | G | Yes |
| 005 | A03 | 22543857 | C | T | Yes |
| 006 | A03 | 8450966 | A | T | Yes |
| 007 | A05 | 766233 | C | A | Yes |
| 008 | A10 | 111125064 | A | G | Yes |
| 009 | A10 | 111437317 | G | A | Yes |
| 010 | A10 | 112338179 | A | T | Yes |
| 011 | A10 | 112651380 | T | G | Yes |
| 012 | A10 | 112940238 | G | A | Yes |
| 013 | A10 | 113053553 | G | A | Yes |
| 014 | A11 | 30523772 | A | G | Yes |
| 015 | A12 | 87529643 | A | G | Yes |
| 016 | A13 | 103027858 | C | A | Yes |
| 017 | D02 | 4110390 | T | G | Yes |
| 018 | D03 | 15331946 | G | A | Yes |
| 019 | D03 | 31088497 | C | T | Yes |
| 020 | D03 | 34610610 | A | G | Yes |
| 021 | D03 | 39193206 | C | T | Yes |
| 022 | D03 | 39600506 | A | T | Yes |
| 023 | D03 | 40070059 | G | A | Yes |
| 024 | D03 | 43229382 | A | G | Yes |
| 025 | D03 | 43511381 | A | G | Yes |
| 026 | D03 | 43821808 | A | G | Yes |
| 027 | D03 | 44126802 | T | C | Yes |
| 028 | D03 | 45101555 | G | A | Yes |
| 029 | D03 | 45114135 | A | G | Yes |
| 030 | D03 | 45162922 | A | T | Yes |
| 031 | D03 | 45219545 | C | T | Yes |
| 032 | D03 | 45904637 | A | C | Yes |
| 033 | D03 | 46030279 | A | C | Yes |
| 034 | D04 | 53296491 | T | A | Yes |
| 035 | D04 | 53338317 | A | G | Yes |
| 036 | D04 | 55873730 | G | T | Yes |
| 037 | D04 | 55895101 | A | T | Yes |
| 038 | D06 | 11366732 | G | T | Yes |
| 039 | D09 | 7626032 | T | C | Yes |
| 040 | D10 | 5613642 | C | T | Yes |
| 041 | D10 | 65188256 | T | C | Yes |
| 042 | D11 | 171062 | T | C | Yes |
| 043 | D11 | 23848335 | T | C | Yes |
| 044 | D11 | 23889334 | T | C | Yes |
| 045 | D11 | 23957256 | A | T | Yes |
| 046 | D11 | 24042912 | C | T | Yes |
| 047 | D11 | 9255660 | C | T | Yes |
| 048 | D13 | 4503413 | G | A | Yes |
| 049 | A02 | 103887986 | G | A | Yes |
| 050 | A02 | 103969684 | T | C | Yes |
| 051 | A02 | 103980780 | A | G | Yes |
| 052 | A03 | 7639601 | A | G | Yes |
| 053 | A04 | 4584038 | G | A | Yes |
| 054 | A04 | 4584101 | C | T | Yes |
| 055 | A05 | 69623179 | C | T | Yes |
| 056 | A06 | 28180129 | G | G | Yes |
| 057 | A06 | 28338559 | T | G | Yes |
| 058 | A06 | 28760155 | A | G | Yes |
| 059 | A06 | 37577336 | T | C | Yes |
| 060 | A07 | 10774519 | G | A | Yes |
| 061 | A07 | 89225727 | T | C | Yes |
| 062 | A07 | 90178382 | T | C | Yes |
| 063 | A07 | 90300535 | G | T | Yes |
| 064 | A07 | 90432981 | T | C | Yes |
| 065 | A07 | 90438470 | G | A | Yes |
| 066 | A07 | 90497386 | C | T | Yes |
| 067 | A09 | 4830640 | G | A | Yes |
| 068 | A09 | 77528103 | G | T | Yes |
| 069 | A10 | 109602998 | G | A | Yes |
| 070 | A10 | 112187864 | C | A | Yes |
| 071 | A10 | 112213784 | T | C | Yes |
| 072 | A10 | 75431311 | A | G | Yes |
| 073 | A10 | 75517245 | C | G | Yes |
| 074 | A10 | 7883291 | T | C | Yes |
| 075 | A11 | 114890178 | C | T | Yes |
| 076 | A11 | 15811782 | G | A | Yes |
| 077 | A11 | 23909944 | C | | Yes |
| 078 | A11 | 360614 | A | C | Yes |
| 079 | A11 | 90244406 | C | A | Yes |
| 080 | A12 | 57229716 | A | G | Yes |
| 081 | A12 | 88694205 | C | T | Yes |
| 082 | A12 | 90492050 | C | T | Yes |
| 083 | A12 | 98576530 | C | T | Yes |
| 084 | A13 | 105476632 | G | T | Yes |
| 085 | A13 | 105477213 | G | A | Yes |
| 086 | A13 | 105486165 | G | A | Yes |
| 087 | A13 | 105603690 | T | C | Yes |
| 088 | D01 | 64087028 | G | C | Yes |
| 089 | D02 | 1013903 | T | C | Yes |
| 090 | D02 | 1396386 | T | C | Yes |
| 091 | D03 | 43272859 | G | C | Yes |
| 092 | D03 | 43289074 | A | T | Yes |
| 093 | D05 | 23684485 | C | A | Yes |
| 094 | D05 | 5720265 | G | T | Yes |
| 095 | D05 | 57485276 | C | A | Yes |
| 096 | D05 | 58079007 | G | A | Yes |
| 097 | D05 | 58177617 | C | T | Yes |
| 098 | D06 | 12003138 | G | A | Yes |
| 099 | D06 | 17773574 | A | G | Yes |
| 100 | D06 | 60207647 | G | C | Yes |
| 101 | D06 | 8776619 | G | | Yes |
| 102 | D07 | 19075384 | A | G | Yes |
| 103 | D07 | 51856497 | T | C | Yes |
| 104 | D07 | 6729179 | A | C | Yes |
| 105 | D08 | 2896926 | A | C | Yes |
| 106 | D08 | 2901847 | A | G | Yes |
| 107 | D08 | 2904400 | A | G | Yes |
| 108 | D08 | 5505113 | G | T | Yes |
| 109 | D08 | 63065133 | G | C | Yes |
| 110 | D09 | 6549285 | G | A | Yes |
| 111 | D10 | 2524436 | C | A | Yes |
| 112 | D11 | 57041068 | C | T | Yes |
| 113 | D11 | 58261390 | A | C | Yes |
| 114 | D11 | 64752270 | C | A | Yes |
| 115 | D11 | 64756382 | T | C | Yes |
| 116 | D11 | 64904998 | G | C | Yes |
| 117 | D11 | 64926164 | C | T | Yes |
| 118 | D11 | 64930470 | T | C | Yes |
| 119 | D11 | 64937064 | G | C | Yes |
| 120 | D11 | 65065455 | C | A | Yes |
| 121 | D12 | 53158662 | G | A | Yes |
| 122 | D12 | 60059695 | T | C | Yes |
| 123 | D13 | 1774748 | C | T | Yes |
| 124 | D13 | 229536 | C | G | Yes |
| 125 | D13 | 45090184 | T | G | Yes |
| 126 | D13 | 53340754 | A | C | Yes |
| 127 | D13 | 8371110 | A | C | Yes |
| 128 | seq1 | 39098148 | T | A | Yes |
| 129 | seq1 | 39098165 | A | C | Yes |
| 130 | seq1 | 39098173 | G | C | Yes |
| 131 | seq1 | 9660644 | C | G | Yes |
| 132 | A01 | 2102346 | T | C | Yes |
| 133 | A01 | 8820452 | A | C | Yes |
| 134 | A02 | 1689682 | C | T | Yes |
| 135 | A03 | 51260473 | T | A | Yes |
| 136 | A04 | 6518897 | C | T | Yes |
| 137 | A05 | 11730945 | G | A | Yes |
| 138 | A05 | 75751203 | T | C | Yes |
| 139 | A06 | 8750649 | T | C | Yes |
| 140 | A07 | 1032070 | A | C | Yes |
| 141 | A07 | 1032071 | G | T | Yes |
| 142 | A07 | 12032478 | C | T | Yes |
| 143 | A07 | 95507309 | C | G | Yes |
| 144 | A07 | 989813 | T | A | Yes |
| 145 | A08 | 117118370 | A | G | Yes |
| 146 | A08 | 119190686 | T | C | Yes |
| 147 | A08 | 124176460 | A | G | Yes |
| 148 | A08 | 6187342 | T | C | Yes |
| 149 | A08 | 97074019 | C | T | Yes |
| 150 | A09 | 13706113 | T | C | Yes |
| 151 | A10 | 109236405 | A | G | Yes |
| 152 | A10 | 114220963 | C | T | Yes |
| 153 | A12 | 94150095 | C | T | Yes |
| 154 | A13 | 106412709 | C | T | Yes |
| 155 | A13 | 109879969 | C | T | Yes |
| 156 | A13 | 6433021 | T | C | Yes |
| 157 | D01 | 1150200 | T | A | Yes |
| 158 | D01 | 18933398 | C | T | Yes |
| 159 | D01 | 600285 | C | T | Yes |
| 160 | D02 | 12661527 | T | C | Yes |
| 161 | D02 | 1920733 | C | G | Yes |
| 162 | D02 | 1946913 | A | G | Yes |
| 163 | D02 | 44762375 | T | C | Yes |
| 164 | D02 | 6392666 | G | A | Yes |
| 165 | D02 | 68951786 | C | A | Yes |
| 166 | D03 | 2487227 | A | G | Yes |
| 167 | D04 | 1347244 | A | G | Yes |
| 168 | D04 | 52647897 | A | G | Yes |
| 169 | D05 | 26764654 | C | T | Yes |
| 170 | D05 | 45810951 | A | G | Yes |
| 171 | D06 | 1497967 | G | A | Yes |
| 172 | D06 | 1624645 | A | C | Yes |
| 173 | D06 | 54504155 | A | T | Yes |
| 174 | D06 | 59180787 | G | A | Yes |
| 175 | D06 | 61085437 | T | A | Yes |
| 176 | D07 | 46303166 | A | G | Yes |
| 177 | D08 | 61439658 | G | A | Yes |
| 178 | D08 | 68631323 | C | T | Yes |
| 179 | D08 | 9335830 | C | T | Yes |
| 180 | D09 | 2749569 | A | C | Yes |
| 181 | D09 | 38068242 | T | C | Yes |
| 182 | D11 | 1280801 | G | A | Yes |
| 183 | D12 | 15727440 | T | C | Yes |
| 184 | D12 | 58551591 | C | T | Yes |
| 185 | D12 | 7113678 | G | A | Yes |
| 186 | D13 | 59655465 | T | G | Yes |
| 187 | D13 | 60649746 | G | A | Yes |
| 188 | A01 | 110967771 | A | C | Yes |
| 189 | A02 | 107361717 | T | A | Yes |
| 190 | A02 | 1707434 | G | A | Yes |
| 191 | A02 | 26551420 | G | A | Yes |
| 192 | A05 | 14504864 | C | T | Yes |
| 193 | A05 | 14504889 | A | G | Yes |
| 194 | A05 | 15429410 | C | G | Yes |
| 195 | A07 | 42902490 | C | G | Yes |
| 196 | A07 | 90603512 | T | G | Yes |
| 197 | A07 | 90783146 | G | A | Yes |
| 198 | A08 | 112071927 | C | T | Yes |
| 199 | A10 | 110613300 | T | C | Yes |
| 200 | A11 | 104539963 | T | C | Yes |
| 201 | D02 | 1913626 | A | G | Yes |
| 202 | D02 | 1915388 | T | C | Yes |
| 203 | D02 | 6362048 | C | T | Yes |
| 204 | D04 | 1546736 | T | A | Yes |
| 205 | D05 | 14240130 | T | G | Yes |
| 206 | D06 | 45390691 | C | T | Yes |
| 207 | D06 | 46672551 | C | T | Yes |
| 208 | D08 | 18841330 | A | C | Yes |
| 209 | D11 | 20428933 | A | T | Yes |
| 210 | A01 | 114857126 | G | A | Yes |
| 211 | A01 | 115721972 | A | G | Yes |
| 212 | A01 | 117760175 | T | C | Yes |
| 213 | A01 | 117760658 | T | A | Yes |
| 214 | A02 | 106224475 | A | G | Yes |
| 215 | A02 | 3111851 | A | G | Yes |
| 216 | A03 | 110233891 | A | G | Yes |
| 217 | A04 | 67550448 | A | T | Yes |
| 218 | A04 | 86560855 | C | T | Yes |
| 219 | A04 | 86568861 | C | T | Yes |
| 220 | A05 | 105715349 | A | G | Yes |
| 221 | A05 | 15876258 | T | C | Yes |
| 222 | A05 | 15876308 | C | G | Yes |
| 223 | A05 | 1874595 | A | G | Yes |
| 224 | A05 | 2124121 | C | T | Yes |
| 225 | A05 | 22775565 | G | A | Yes |
| 226 | A05 | 22924264 | T | A | Yes |
| 227 | A05 | 22938538 | A | T | Yes |
| 228 | A05 | 31423050 | T | C | Yes |
| 229 | A06 | 119255407 | C | T | Yes |
| 230 | A06 | 124093748 | C | T | Yes |
| 231 | A06 | 1503775 | C | G | Yes |
| 232 | A07 | 21563371 | C | T | Yes |
| 233 | A07 | 23460168 | C | T | Yes |
| 234 | A07 | 83188617 | G | C | Yes |
| 235 | A07 | 88838111 | C | T | Yes |
| 236 | A07 | 90625054 | C | T | Yes |
| 237 | A07 | 90633925 | C | T | Yes |
| 238 | A07 | 90639206 | T | C | Yes |
| 239 | A08 | 104027372 | A | G | Yes |
| 240 | A08 | 107661059 | T | G | Yes |
| 241 | A08 | 123086263 | C | G | Yes |
| 242 | A08 | 123101111 | T | C | Yes |
| 243 | A08 | 31131479 | C | T | Yes |
| 244 | A08 | 6469161 | T | A | Yes |
| 245 | A09 | 73717101 | C | A | Yes |
| 246 | A10 | 108187386 | C | T | Yes |
| 247 | A10 | 114006682 | G | A | Yes |
| 248 | A10 | 2516523 | C | T | Yes |
| 249 | A10 | 4309496 | C | A | Yes |
| 250 | A11 | 118950829 | A | T | Yes |
| 251 | A11 | 24222 | G | A | Yes |
| 252 | A11 | 9073286 | G | A | Yes |
| 253 | A11 | 99926974 | A | G | Yes |
| 254 | A12 | 104493499 | T | A | Yes |
| 255 | A12 | 95983460 | C | G | Yes |
| 256 | A13 | 100671788 | C | T | Yes |
| 257 | A13 | 104864916 | T | G | Yes |
| 258 | A13 | 107008682 | A | G | Yes |
| 259 | A13 | 5759232 | A | G | Yes |
| 260 | A13 | 68442801 | C | A | Yes |
| 261 | A13 | 89442130 | G | A | Yes |
| 262 | A13 | 98283310 | T | C | Yes |
| 263 | D01 | 19034036 | T | A | Yes |
| 264 | D01 | 54855218 | T | C | Yes |
| 265 | D01 | 54866986 | C | T | Yes |
| 266 | D01 | 54890106 | T | C | Yes |
| 267 | D01 | 54896281 | G | A | Yes |
| 268 | D01 | 57261588 | A | G | Yes |
| 269 | D01 | 58881229 | C | T | Yes |
| 270 | D02 | 51851185 | A | G | Yes |
| 271 | D02 | 52987299 | A | G | Yes |
| 272 | D02 | 53034743 | G | T | Yes |
| 273 | D02 | 53253589 | A | C | Yes |
| 274 | D02 | 54901956 | C | T | Yes |
| 275 | D02 | 63310106 | T | C | Yes |
| 276 | D02 | 6401616 | T | A | Yes |
| 277 | D02 | 68030533 | A | T | Yes |
| 278 | D02 | 7489387 | T | G | Yes |
| 279 | D02 | 7531127 | A | G | Yes |
| 280 | D03 | 2503186 | G | T | Yes |
| 281 | D04 | 52350838 | T | G | Yes |
| 282 | D04 | 6848742 | A | C | Yes |
| 283 | D04 | 6861687 | C | T | Yes |
| 284 | D04 | 851577 | G | A | Yes |
| 285 | D05 | 14718765 | A | G | Yes |
| 286 | D05 | 55154526 | T | C | Yes |
| 287 | D05 | 56805649 | A | G | Yes |
| 288 | D06 | 18372518 | T | A | Yes |
| 289 | D06 | 47311286 | G | T | Yes |
| 290 | D06 | 64318600 | T | C | Yes |
| 291 | D07 | 10130687 | G | C | Yes |
| 292 | D07 | 54754308 | T | C | Yes |
| 293 | D07 | 54818140 | G | A | Yes |
| 294 | D07 | 54821539 | T | A | Yes |
| 295 | D08 | 2748641 | C | T | Yes |
| 296 | D08 | 430878 | C | A | Yes |
| 297 | D08 | 4868113 | C | T | Yes |
| 298 | D08 | 608978 | C | T | Yes |
| 299 | D09 | 49092148 | A | G | Yes |
| 300 | D09 | 6069425 | C | T | Yes |
| 301 | D10 | 63443095 | G | C | Yes |
| 302 | D10 | 63918764 | C | T | Yes |
| 303 | D10 | 64184291 | A | T | Yes |
| 304 | D10 | 826050 | A | G | Yes |
| 305 | D11 | 10658879 | T | C | Yes |
| 306 | D11 | 13890576 | T | C | Yes |
| 307 | D11 | 21988164 | G | A | Yes |
| 308 | D11 | 23924389 | T | A | Yes |
| 309 | D11 | 23924409 | C | T | Yes |
| 310 | D11 | 23944152 | C | T | Yes |
| 311 | D11 | 24002127 | A | G | Yes |
| 312 | D11 | 24004889 | C | T | Yes |
| 313 | D11 | 24034422 | G | C | Yes |
| 314 | D11 | 24036519 | A | G | Yes |
| 315 | D11 | 3229941 | A | G | Yes |
| 316 | D11 | 3230659 | C | T | Yes |
| 317 | D11 | 62899476 | G | A | Yes |
| 318 | D12 | 1201966 | C | A | Yes |
| 319 | D12 | 2093018 | T | G | Yes |
| 320 | D12 | 2108978 | A | G | Yes |
| 321 | D12 | 2237848 | G | A | Yes |
| 322 | D12 | 49521512 | A | G | Yes |
| 323 | D12 | 49927212 | G | A | Yes |
| 324 | D12 | 53455112 | T | A | Yes |
| 325 | D12 | 55839588 | C | T | Yes |
| 326 | D12 | 61139512 | A | C | Yes |
| 327 | D12 | 61140550 | C | T | Yes |
| 328 | D13 | 44115715 | T | G | Yes |
| 329 | D13 | 45131566 | A | G | Yes |
| 330 | A01 | 102077466 | C | T | No |
| 331 | A01 | 103023924 | G | A | No |
| 332 | A01 | 109299115 | A | G | No |
| 333 | A01 | 110128160 | A | G | No |
| 334 | A01 | 110687776 | A | T | No |
| 335 | A01 | 113567604 | C | T | No |
| 336 | A01 | 114087981 | G | A | No |
| 337 | A01 | 11692652 | C | T | No |
| 338 | A01 | 17937689 | T | C | No |
| 339 | A01 | 19586649 | T | C | No |
| 340 | A01 | 20011715 | C | T | No |
| 341 | A01 | 23552093 | C | T | No |
| 342 | A01 | 25130938 | T | C | No |
| 343 | A01 | 29630645 | A | G | No |
| 344 | A01 | 30304909 | A | G | No |
| 345 | A01 | 30655283 | T | C | No |
| 346 | A01 | 31298172 | T | A | No |
| 347 | A01 | 37097863 | G | A | No |
| 348 | A01 | 39816020 | G | C | No |
| 349 | A01 | 44008051 | G | A | No |
| 350 | A01 | 45886993 | C | T | No |
| 351 | A01 | 48030807 | G | A | No |
| 352 | A01 | 50753667 | C | A | No |
| 353 | A01 | 52461718 | G | C | No |
| 354 | A01 | 53550328 | A | G | No |
| 355 | A01 | 55361298 | T | C | No |
| 356 | A01 | 5665093 | A | G | No |
| 357 | A01 | 59440674 | A | G | No |
| 358 | A01 | 60660225 | T | C | No |
| 359 | A01 | 62406568 | T | C | No |
| 360 | A01 | 69399954 | A | G | No |
| 361 | A01 | 72356977 | A | G | No |
| 362 | A01 | 73066978 | C | T | No |
| 363 | A01 | 83056539 | G | C | No |
| 364 | A01 | 9307886 | T | C | No |
| 365 | A01 | 94433050 | C | G | No |
| 366 | A01 | 97114671 | G | A | No |
| 367 | A01 | 9928461 | A | G | No |
| 368 | A01 | 9992909 | T | C | No |
| 369 | A02 | 104702522 | C | G | No |
| 370 | A02 | 106564817 | G | A | No |
| 371 | A02 | 126465 | A | G | No |
| 372 | A02 | 18537125 | A | G | No |
| 373 | A02 | 19418124 | T | C | No |
| 374 | A02 | 3608260 | T | A | No |
| 375 | A02 | 3753565 | C | G | No |
| 376 | A02 | 39122586 | T | C | No |
| 377 | A02 | 44761669 | C | A | No |
| 378 | A02 | 44836527 | T | C | No |
| 379 | A02 | 52198772 | A | G | No |
| 380 | A02 | 63519897 | A | G | No |
| 381 | A02 | 64573030 | C | T | No |
| 382 | A02 | 64810766 | A | G | No |
| 383 | A02 | 71898039 | G | C | No |
| 384 | A02 | 76764218 | C | A | No |
| 385 | A02 | 79665360 | C | T | No |
| 386 | A02 | 82773518 | A | G | No |
| 387 | A02 | 8356092 | C | T | No |
| 388 | A02 | 95037408 | T | G | No |
| 389 | A03 | 10432099 | T | C | No |
| 390 | A03 | 104900576 | A | C | No |
| 391 | A03 | 106410955 | G | A | No |
| 392 | A03 | 16588547 | T | C | No |
| 393 | A03 | 35348610 | T | C | No |
| 394 | A03 | 36068818 | T | C | No |
| 395 | A03 | 41629111 | T | C | No |
| 396 | A03 | 62010341 | C | T | No |
| 397 | A03 | 72339471 | A | G | No |
| 398 | A03 | 81970703 | T | C | No |
| 399 | A03 | 91791344 | T | C | No |
| 400 | A03 | 93575802 | T | C | No |
| 401 | A03 | 93642770 | G | T | No |
| 402 | A03 | 95039694 | T | C | No |
| 403 | A03 | 95047213 | A | G | No |
| 404 | A03 | 95068176 | A | C | No |
| 405 | A03 | 95073577 | T | A | No |
| 406 | A03 | 95086199 | C | T | No |
| 407 | A03 | 95090581 | A | G | No |
| 408 | A03 | 95091209 | G | A | No |
| 409 | A03 | 97554243 | A | T | No |
| 410 | A04 | 11004835 | T | G | No |
| 411 | A04 | 11910166 | C | T | No |
| 412 | A04 | 12158377 | C | T | No |
| 413 | A04 | 12243883 | A | G | No |
| 414 | A04 | 13827618 | T | C | No |
| 415 | A04 | 16789748 | A | G | No |
| 416 | A04 | 1886870 | C | T | No |
| 417 | A04 | 1958101 | A | G | No |
| 418 | A04 | 1969979 | T | C | No |
| 419 | A04 | 22382054 | C | G | No |
| 420 | A04 | 36328459 | G | A | No |
| 421 | A04 | 37588019 | A | G | No |
| 422 | A04 | 55428635 | G | A | No |
| 423 | A04 | 61481730 | C | T | No |
| 424 | A04 | 69596220 | G | A | No |
| 425 | A04 | 7163673 | C | G | No |
| 426 | A04 | 7164063 | C | T | No |
| 427 | A04 | 81630879 | T | C | No |
| 428 | A04 | 83437396 | A | G | No |
| 429 | A04 | 84817926 | T | A | No |
| 430 | A05 | 101965827 | A | G | No |
| 431 | A05 | 102937436 | T | C | No |
| 432 | A05 | 106162489 | C | T | No |
| 433 | A05 | 1273243 | T | G | No |
| 434 | A05 | 24223452 | C | T | No |
| 435 | A05 | 28843012 | G | A | No |
| 436 | A05 | 36231152 | A | G | No |
| 437 | A05 | 36585565 | T | G | No |
| 438 | A05 | 39529362 | C | T | No |
| 439 | A05 | 40529916 | T | C | No |
| 440 | A05 | 54824863 | C | T | No |
| 441 | A05 | 59499071 | G | A | No |
| 442 | A05 | 75568141 | A | G | No |
| 443 | A05 | 75568470 | A | G | No |
| 444 | A05 | 78899507 | G | A | No |
| 445 | A05 | 94441223 | C | G | No |
| 446 | A05 | 94467545 | G | T | No |
| 447 | A05 | 96112268 | T | C | No |
| 448 | A05 | 97278925 | G | T | No |
| 449 | A05 | 98875724 | G | T | No |
| 450 | A06 | 100673641 | T | C | No |
| 451 | A06 | 119009915 | A | G | No |
| 452 | A06 | 120558212 | G | A | No |
| 453 | A06 | 12521911 | A | G | No |
| 454 | A06 | 1289926 | T | C | No |
| 455 | A06 | 14409659 | T | C | No |
| 456 | A06 | 19263878 | T | C | No |
| 457 | A06 | 19510906 | A | G | No |
| 458 | A06 | 24921227 | T | G | No |
| 459 | A06 | 25685023 | C | T | No |
| 460 | A06 | 30796846 | T | C | No |
| 461 | A06 | 32683074 | T | C | No |
| 462 | A06 | 32798852 | G | A | No |
| 463 | A06 | 34369034 | T | C | No |
| 464 | A06 | 36798412 | T | C | No |
| 465 | A06 | 46083287 | G | A | No |
| 466 | A06 | 46958830 | A | C | No |
| 467 | A06 | 47635932 | A | G | No |
| 468 | A06 | 47877963 | A | G | No |
| 469 | A06 | 5322494 | A | C | No |
| 470 | A06 | 72157456 | T | C | No |
| 471 | A06 | 73439358 | G | A | No |
| 472 | A06 | 75814615 | C | T | No |
| 473 | A06 | 78918757 | A | G | No |
| 474 | A06 | 80502017 | G | C | No |
| 475 | A06 | 80669852 | T | C | No |
| 476 | A06 | 80914576 | T | C | No |
| 477 | A06 | 83069476 | A | G | No |
| 478 | A06 | 83420195 | A | G | No |
| 479 | A06 | 83713547 | A | G | No |
| 480 | A06 | 89002323 | T | C | No |
| 481 | A06 | 89566493 | T | C | No |
| 482 | A06 | 90969355 | A | G | No |
| 483 | A06 | 93187658 | T | C | No |
| 484 | A06 | 95478943 | T | C | No |
| 485 | A06 | 973530 | C | T | No |
| 486 | A07 | 11906060 | T | C | No |
| 487 | A07 | 11915754 | C | T | No |
| 488 | A07 | 12320575 | T | C | No |
| 489 | A07 | 18648297 | G | A | No |
| 490 | A07 | 21310224 | T | C | No |
| 491 | A07 | 21785923 | A | G | No |
| 492 | A07 | 25884125 | G | A | No |
| 493 | A07 | 29041927 | C | T | No |
| 494 | A07 | 36875269 | C | G | No |
| 495 | A07 | 38342570 | T | C | No |
| 496 | A07 | 40698615 | G | A | No |
| 497 | A07 | 42581937 | T | C | No |
| 498 | A07 | 43180984 | A | G | No |
| 499 | A07 | 43810960 | A | G | No |
| 500 | A07 | 43986311 | T | C | No |
| 501 | A07 | 47819857 | A | G | No |
| 502 | A07 | 54517045 | G | A | No |
| 503 | A07 | 64238084 | C | T | No |
| 504 | A07 | 80217572 | C | T | No |
| 505 | A07 | 80388490 | A | T | No |
| 506 | A07 | 82916184 | T | C | No |
| 507 | A07 | 83105438 | G | A | No |
| 508 | A07 | 8422752 | G | C | No |
| 509 | A07 | 89161736 | A | G | No |
| 510 | A07 | 9478030 | A | C | No |
| 511 | A08 | 10429943 | C | G | No |
| 512 | A08 | 106893339 | C | T | No |
| 513 | A08 | 109696323 | G | A | No |
| 514 | A08 | 112025642 | C | T | No |
| 515 | A08 | 113464785 | A | G | No |
| 516 | A08 | 116730869 | A | G | No |
| 517 | A08 | 1713011 | T | C | No |
| 518 | A08 | 19121855 | C | A | No |
| 519 | A08 | 20388949 | G | A | No |
| 520 | A08 | 25642734 | C | T | No |
| 521 | A08 | 29214900 | C | T | No |
| 522 | A08 | 29234582 | A | C | No |
| 523 | A08 | 29239926 | T | C | No |
| 524 | A08 | 29243822 | A | G | No |
| 525 | A08 | 4039263 | C | T | No |
| 526 | A08 | 48463216 | T | C | No |
| 527 | A08 | 55388879 | C | T | No |
| 528 | A08 | 56754026 | A | G | No |
| 529 | A08 | 63452446 | G | A | No |
| 530 | A08 | 64485512 | G | A | No |
| 531 | A08 | 78341697 | C | T | No |
| 532 | A08 | 89152238 | A | G | No |
| 533 | A08 | 89152667 | T | C | No |
| 534 | A08 | 93471156 | A | T | No |
| 535 | A08 | 99343910 | A | G | No |
| 536 | A09 | 12673218 | T | C | No |
| 537 | A09 | 12998204 | C | T | No |
| 538 | A09 | 14103382 | C | T | No |
| 539 | A09 | 20823587 | T | A | No |
| 540 | A09 | 20844042 | G | A | No |
| 541 | A09 | 23502455 | A | C | No |
| 542 | A09 | 25275518 | T | G | No |
| 543 | A09 | 31554465 | C | T | No |
| 544 | A09 | 40968413 | A | G | No |
| 545 | A09 | 42870985 | A | G | No |
| 546 | A09 | 45777 | C | A | No |
| 547 | A09 | 47518943 | G | A | No |
| 548 | A09 | 51396443 | G | A | No |
| 549 | A09 | 55493894 | C | G | No |
| 550 | A09 | 61184216 | A | G | No |
| 551 | A09 | 61363297 | G | T | No |
| 552 | A09 | 65591384 | T | C | No |
| 553 | A09 | 67674689 | G | T | No |
| 554 | A09 | 76025039 | G | A | No |
| 555 | A10 | 10543466 | A | G | No |
| 556 | A10 | 106581756 | T | C | No |
| 557 | A10 | 107143359 | T | G | No |
| 558 | A10 | 107787459 | G | A | No |
| 559 | A10 | 10945265 | A | C | No |
| 560 | A10 | 112338443 | T | A | No |
| 561 | A10 | 15068940 | G | A | No |
| 562 | A10 | 18866303 | A | C | No |
| 563 | A10 | 27447943 | A | G | No |
| 564 | A10 | 3130405 | C | T | No |
| 565 | A10 | 3310056 | T | C | No |
| 566 | A10 | 45696050 | C | A | No |
| 567 | A10 | 48324687 | G | A | No |
| 568 | A10 | 48366731 | A | G | No |
| 569 | A10 | 56444175 | C | T | No |
| 570 | A10 | 6404617 | T | C | No |
| 571 | A10 | 6555180 | A | G | No |
| 572 | A10 | 6738311 | A | C | No |
| 573 | A10 | 68078336 | A | G | No |
| 574 | A10 | 76202810 | T | C | No |
| 575 | A10 | 85468645 | A | G | No |
| 576 | A11 | 11016688 | T | C | No |
| 577 | A11 | 11441789 | A | G | No |
| 578 | A11 | 1394259 | C | G | No |
| 579 | A11 | 16077265 | G | A | No |
| 580 | A11 | 18005388 | A | G | No |
| 581 | A11 | 23540980 | C | T | No |
| 582 | A11 | 25232174 | C | A | No |
| 583 | A11 | 25233949 | T | C | No |
| 584 | A11 | 30735652 | A | G | No |
| 585 | A11 | 31753695 | A | G | No |
| 586 | A11 | 35871174 | A | C | No |
| 587 | A11 | 37921426 | C | A | No |
| 588 | A11 | 52514719 | T | C | No |
| 589 | A11 | 55123151 | A | G | No |
| 590 | A11 | 56784796 | T | C | No |
| 591 | A11 | 62701332 | T | C | No |
| 592 | A11 | 63434388 | A | G | No |
| 593 | A11 | 66244941 | A | G | No |
| 594 | A11 | 67626090 | T | C | No |
| 595 | A11 | 67630369 | G | C | No |
| 596 | A11 | 68182818 | A | G | No |
| 597 | A11 | 68183232 | C | T | No |
| 598 | A11 | 70719189 | A | G | No |
| 599 | A11 | 77534048 | C | T | No |
| 600 | A11 | 82139694 | G | A | No |
| 601 | A11 | 85424243 | T | C | No |
| 602 | A11 | 89746643 | A | T | No |
| 603 | A11 | 90149203 | C | T | No |
| 604 | A11 | 92066056 | T | C | No |
| 605 | A11 | 92429725 | C | T | No |
| 606 | A11 | 95752317 | A | T | No |
| 607 | A11 | 98573925 | A | G | No |
| 608 | A12 | 10226218 | G | A | No |
| 609 | A12 | 11396410 | G | A | No |
| 610 | A12 | 11429480 | G | A | No |
| 611 | A12 | 12115755 | C | T | No |
| 612 | A12 | 16073340 | A | G | No |
| 613 | A12 | 17820382 | G | T | No |
| 614 | A12 | 22544556 | T | G | No |
| 615 | A12 | 24422845 | T | C | No |
| 616 | A12 | 3233765 | A | G | No |
| 617 | A12 | 36619695 | G | A | No |
| 618 | A12 | 40703533 | G | A | No |
| 619 | A12 | 4152128 | A | G | No |
| 620 | A12 | 44104522 | G | T | No |
| 621 | A12 | 44388198 | A | G | No |
| 622 | A12 | 44629878 | T | A | No |
| 623 | A12 | 5091648 | T | A | No |
| 624 | A12 | 52831490 | G | C | No |
| 625 | A12 | 60989615 | A | C | No |
| 626 | A12 | 6396346 | A | G | No |
| 627 | A12 | 68296841 | G | A | No |
| 628 | A12 | 7194840 | G | A | No |
| 629 | A12 | 77899237 | C | T | No |
| 630 | A12 | 7810671 | C | G | No |
| 631 | A12 | 7969574 | A | G | No |
| 632 | A12 | 80173437 | G | A | No |
| 633 | A13 | 100127785 | G | A | No |
| 634 | A13 | 101918312 | G | A | No |
| 635 | A13 | 17036027 | A | C | No |
| 636 | A13 | 17036590 | C | T | No |
| 637 | A13 | 21731709 | A | G | No |
| 638 | A13 | 23198397 | T | C | No |
| 639 | A13 | 27322121 | A | G | No |
| 640 | A13 | 278763 | T | G | No |
| 641 | A13 | 35844566 | G | A | No |
| 642 | A13 | 45195746 | A | G | No |
| 643 | A13 | 45220863 | T | C | No |
| 644 | A13 | 45223069 | A | G | No |
| 645 | A13 | 45229632 | C | G | No |
| 646 | A13 | 45230181 | G | A | No |
| 647 | A13 | 45238518 | T | A | No |
| 648 | A13 | 45240195 | A | G | No |
| 649 | A13 | 50036635 | C | G | No |
| 650 | A13 | 59746791 | T | C | No |
| 651 | A13 | 7768028 | C | T | No |
| 652 | A13 | 77893733 | G | A | No |
| 653 | A13 | 78230319 | G | A | No |
| 654 | A13 | 79597429 | A | G | No |
| 655 | A13 | 80392586 | T | C | No |
| 656 | A13 | 80452058 | A | G | No |
| 657 | A13 | 80730886 | C | A | No |
| 658 | A13 | 81306936 | C | T | No |
| 659 | A13 | 83761879 | T | C | No |
| 660 | A13 | 89445949 | A | G | No |
| 661 | A13 | 9366668 | A | T | No |
| 662 | A13 | 94821021 | C | T | No |
| 663 | A13 | 94837745 | C | T | No |
| 664 | A13 | 94898464 | G | T | No |
| 665 | A13 | 96661617 | A | G | No |
| 666 | A13 | 97785453 | C | T | No |
| 667 | D01 | 24764505 | A | T | No |
| 668 | D01 | 24767839 | T | C | No |
| 669 | D01 | 31464245 | G | T | No |
| 670 | D01 | 32265626 | A | G | No |
| 671 | D01 | 32543583 | T | C | No |
| 672 | D01 | 33576723 | A | G | No |
| 673 | D01 | 37246475 | G | A | No |
| 674 | D01 | 38589185 | C | A | No |
| 675 | D01 | 40297859 | T | C | No |
| 676 | D01 | 42570072 | C | A | No |
| 677 | D01 | 46056348 | C | A | No |
| 678 | D01 | 54437017 | G | T | No |
| 679 | D01 | 54531974 | A | G | No |
| 680 | D01 | 54532596 | A | C | No |
| 681 | D01 | 54841313 | C | T | No |
| 682 | D01 | 56204975 | T | C | No |
| 683 | D01 | 56387888 | T | A | No |
| 684 | D01 | 59941820 | C | T | No |
| 685 | D01 | 63516128 | A | G | No |
| 686 | D01 | 9090589 | A | T | No |
| 687 | D02 | 17045564 | T | A | No |
| 688 | D02 | 18307420 | C | A | No |
| 689 | D02 | 18765233 | A | G | No |
| 690 | D02 | 19924810 | G | A | No |
| 691 | D02 | 23266296 | G | A | No |
| 692 | D02 | 29523923 | T | A | No |
| 693 | D02 | 30504620 | G | T | No |
| 694 | D02 | 38335405 | A | G | No |
| 695 | D02 | 45180077 | T | A | No |
| 696 | D02 | 4665984 | G | A | No |
| 697 | D02 | 50316439 | A | C | No |
| 698 | D02 | 50316584 | A | G | No |
| 699 | D02 | 54044417 | A | G | No |
| 700 | D02 | 57420904 | G | A | No |
| 701 | D02 | 57735413 | T | C | No |
| 702 | D02 | 59149744 | A | C | No |
| 703 | D02 | 60903663 | A | G | No |
| 704 | D02 | 64071331 | A | G | No |
| 705 | D02 | 64105609 | T | C | No |
| 706 | D02 | 652122 | G | A | No |
| 707 | D02 | 69573139 | T | C | No |
| 708 | D03 | 10851700 | G | C | No |
| 709 | D03 | 12601043 | T | C | No |
| 710 | D03 | 19246208 | T | C | No |
| 711 | D03 | 25627391 | C | T | No |
| 712 | D03 | 25646216 | C | T | No |
| 713 | D03 | 27701555 | C | T | No |
| 714 | D03 | 31735979 | T | C | No |
| 715 | D03 | 34571433 | G | T | No |
| 716 | D03 | 36559975 | C | T | No |
| 717 | D03 | 4447807 | G | A | No |
| 718 | D03 | 46722299 | T | C | No |
| 719 | D03 | 47897510 | G | A | No |
| 720 | D03 | 48891235 | A | G | No |
| 721 | D03 | 50598904 | G | T | No |
| 722 | D03 | 5253112 | T | G | No |
| 723 | D03 | 5254940 | A | C | No |
| 724 | D03 | 6554186 | A | G | No |
| 725 | D03 | 6884462 | G | T | No |
| 726 | D03 | 7213302 | T | C | No |
| 727 | D03 | 8405927 | A | G | No |
| 728 | D04 | 19963729 | C | G | No |
| 729 | D04 | 25993830 | C | T | No |
| 730 | D04 | 28820458 | C | T | No |
| 731 | D04 | 34164246 | A | G | No |
| 732 | D04 | 34164765 | T | C | No |
| 733 | D04 | 34168061 | T | C | No |
| 734 | D04 | 34170181 | T | G | No |
| 735 | D04 | 34186357 | A | G | No |
| 736 | D04 | 34188285 | T | C | No |
| 737 | D04 | 34209861 | G | T | No |
| 738 | D04 | 34212452 | T | C | No |
| 739 | D04 | 4477447 | G | A | No |
| 740 | D04 | 50668212 | C | T | No |
| 741 | D04 | 54749560 | G | T | No |
| 742 | D04 | 55088982 | T | G | No |
| 743 | D04 | 6405687 | C | T | No |
| 744 | D04 | 9885360 | G | C | No |
| 745 | D05 | 10111963 | A | G | No |
| 746 | D05 | 22301632 | T | C | No |
| 747 | D05 | 22581979 | C | A | No |
| 748 | D05 | 44163822 | C | G | No |
| 749 | D05 | 4754359 | G | A | No |
| 750 | D05 | 50425541 | G | A | No |
| 751 | D05 | 54319989 | C | T | No |
| 752 | D05 | 54328389 | G | A | No |
| 753 | D05 | 60809038 | C | G | No |
| 754 | D05 | 63575099 | G | A | No |
| 755 | D06 | 120461 | T | C | No |
| 756 | D06 | 15573192 | C | A | No |
| 757 | D06 | 24091586 | T | G | No |
| 758 | D06 | 32611737 | T | C | No |
| 759 | D06 | 37123528 | A | G | No |
| 760 | D06 | 39543027 | T | C | No |
| 761 | D06 | 40371530 | A | C | No |
| 762 | D06 | 40976675 | T | G | No |
| 763 | D06 | 4587872 | A | G | No |
| 764 | D06 | 459987 | A | G | No |
| 765 | D06 | 46781842 | A | G | No |
| 766 | D06 | 51896336 | G | A | No |
| 767 | D06 | 52879640 | A | G | No |
| 768 | D06 | 8300282 | C | A | No |
| 769 | D06 | 8301102 | A | G | No |
| 770 | D06 | 8302085 | A | T | No |
| 771 | D06 | 8316146 | C | G | No |
| 772 | D06 | 8316953 | A | G | No |
| 773 | D06 | 8317874 | T | G | No |
| 774 | D06 | 9134552 | G | A | No |
| 775 | D06 | 984077 | G | C | No |
| 776 | D07 | 12186527 | T | A | No |
| 777 | D07 | 18817779 | C | T | No |
| 778 | D07 | 19736648 | A | T | No |
| 779 | D07 | 21947521 | T | C | No |
| 780 | D07 | 23120977 | A | G | No |
| 781 | D07 | 28055711 | G | A | No |
| 782 | D07 | 29109329 | C | A | No |
| 783 | D07 | 29307287 | C | A | No |
| 784 | D07 | 31304191 | T | A | No |
| 785 | D07 | 33903373 | A | G | No |
| 786 | D07 | 35288441 | A | C | No |
| 787 | D07 | 35472043 | G | A | No |
| 788 | D07 | 42317367 | A | G | No |
| 789 | D07 | 48374078 | C | T | No |
| 790 | D07 | 4924881 | T | A | No |
| 791 | D07 | 53499692 | T | C | No |
| 792 | D08 | 10680089 | C | G | No |
| 793 | D08 | 13077583 | T | C | No |
| 794 | D08 | 149324 | C | T | No |
| 795 | D08 | 16413758 | T | A | No |
| 796 | D08 | 1767573 | C | T | No |
| 797 | D08 | 18913124 | T | C | No |
| 798 | D08 | 21560032 | C | T | No |
| 799 | D08 | 22149204 | C | A | No |
| 800 | D08 | 23067189 | T | C | No |
| 801 | D08 | 23093384 | C | A | No |
| 802 | D08 | 31945553 | A | G | No |
| 803 | D08 | 4830232 | G | T | No |
| 804 | D08 | 53958299 | A | G | No |
| 805 | D08 | 54171228 | C | T | No |
| 806 | D08 | 60184589 | T | A | No |
| 807 | D08 | 60980269 | T | G | No |
| 808 | D08 | 64419820 | T | C | No |
| 809 | D08 | 69065625 | C | T | No |
| 810 | D09 | 11775224 | T | C | No |
| 811 | D09 | 13077530 | G | A | No |
| 812 | D09 | 13092466 | G | A | No |
| 813 | D09 | 14556836 | A | G | No |
| 814 | D09 | 14980165 | T | C | No |
| 815 | D09 | 16586783 | G | A | No |
| 816 | D09 | 16915286 | T | G | No |
| 817 | D09 | 29378213 | T | C | No |
| 818 | D09 | 30116231 | T | C | No |
| 819 | D09 | 32511822 | A | G | No |
| 820 | D09 | 35843751 | T | A | No |
| 821 | D09 | 39952298 | A | G | No |
| 822 | D09 | 40931477 | T | G | No |
| 823 | D09 | 5153702 | A | G | No |
| 824 | D09 | 5327090 | A | T | No |
| 825 | D09 | 7625892 | A | G | No |
| 826 | D09 | 8984707 | T | C | No |
| 827 | D10 | 11881833 | G | A | No |
| 828 | D10 | 12641518 | T | C | No |
| 829 | D10 | 13203079 | T | C | No |
| 830 | D10 | 13218360 | A | G | No |
| 831 | D10 | 17236139 | A | C | No |
| 832 | D10 | 21015217 | A | T | No |
| 833 | D10 | 22279984 | T | C | No |
| 834 | D10 | 23857566 | T | C | No |
| 835 | D10 | 27931417 | A | C | No |
| 836 | D10 | 29136434 | A | G | No |
| 837 | D10 | 36608001 | A | G | No |
| 838 | D10 | 5768028 | T | A | No |
| 839 | D10 | 58648569 | T | C | No |
| 840 | D10 | 59110528 | C | T | No |
| 841 | D10 | 59338863 | A | G | No |
| 842 | D10 | 63020858 | A | G | No |
| 843 | D10 | 65084524 | C | T | No |
| 844 | D10 | 7921537 | G | C | No |
| 845 | D10 | 9584269 | C | T | No |
| 846 | D11 | 18266384 | A | G | No |
| 847 | D11 | 19526253 | A | G | No |
| 848 | D11 | 21313603 | T | C | No |
| 849 | D11 | 22813334 | G | C | No |
| 850 | D11 | 22866559 | G | A | No |
| 851 | D11 | 22965595 | T | C | No |
| 852 | D11 | 25045105 | A | G | No |
| 853 | D11 | 26242153 | A | G | No |
| 854 | D11 | 29069201 | A | G | No |
| 855 | D11 | 30858636 | A | G | No |
| 856 | D11 | 31530446 | T | C | No |
| 857 | D11 | 33562213 | G | T | No |
| 858 | D11 | 36259888 | G | C | No |
| 859 | D11 | 4001453 | C | A | No |
| 860 | D11 | 42064410 | C | G | No |
| 861 | D11 | 46502666 | T | C | No |
| 862 | D11 | 49025870 | T | G | No |
| 863 | D11 | 53984557 | A | G | No |
| 864 | D11 | 62552126 | C | T | No |
| 865 | D11 | 63105221 | T | A | No |
| 866 | D12 | 13234020 | C | A | No |
| 867 | D12 | 20303021 | T | G | No |
| 868 | D12 | 20676817 | C | T | No |
| 869 | D12 | 20679556 | G | C | No |
| 870 | D12 | 24086625 | G | A | No |
| 871 | D12 | 31095105 | A | G | No |
| 872 | D12 | 31344122 | G | A | No |
| 873 | D12 | 34101393 | A | G | No |
| 874 | D12 | 36771208 | C | T | No |
| 875 | D12 | 39681819 | T | G | No |
| 876 | D12 | 39682436 | C | T | No |
| 877 | D12 | 40027143 | A | G | No |
| 878 | D12 | 41909287 | A | G | No |
| 879 | D12 | 4257295 | A | G | No |
| 880 | D12 | 4544167 | A | C | No |
| 881 | D12 | 50612739 | T | C | No |
| 882 | D13 | 19377974 | A | G | No |
| 883 | D13 | 2694968 | A | C | No |
| 884 | D13 | 3134551 | A | T | No |
| 885 | D13 | 39085839 | G | A | No |
| 886 | D13 | 42257012 | A | G | No |
| 887 | D13 | 49441679 | A | G | No |
| 888 | D13 | 49745988 | A | G | No |
| 889 | D13 | 55874078 | A | G | No |
| 890 | D13 | 57525048 | T | G | No |
| 891 | D13 | 58479149 | A | G | No |
| 892 | D13 | 63605335 | A | G | No |
| 893 | D13 | 64125386 | T | C | No |
| 894 | D13 | 9724970 | T | A | No |

Where, Ref represents the base type of a SNP locus in the reference genome, and Alt represents the base type of SNP locus in a population.

### Example 2: Use of Low-Density Liquid-Phase Chip for Gossypium hirsutum L. Based on Targeted Capture Sequencing in Genotyping and Detection of Transgenic Components of Gossypium hirsutum L.

200 Gossypium hirsutum L. varieties were used to test the chip. The test results showed that the average locus detection rate of cotton functional marker chip was 94.17%, which met the genotyping test of Gossypium hirsutum L.

The whole experimental process was based on the GenoBaits technical system. The specific experimental process is as follows:

1. The genomic DNAs of samples to be tested were extracted, and a sample library was constructed;

### 1.1 DNA Extraction of the Samples

The DNA of the sample was extracted by a CTAB method.

### 1.2 DNA Quality Inspection of the Samples

The DNA concentration of the test sample was measured by Qubit Fluorometric Quantitation (Thermofisher), and the integrity of the DNA was detected by 1 wt % agarose gel electrophoresis (AGE). The qualified samples were put into a refrigerator at 4° C. for storage and standby.

### 1.3 DNA Fragmentation of the Sample

12 μL of the qualified DNA was placed in a 0.2 μL PCR tube, the tube was placed in an ultrasonic crusher to randomly physically crush the DNA, with the fragment being crushed to 200-400 bp.

### 1.4 End Repair of the Samples

4 μL of GenoBaits End Repair Buffer (genobaits, Shijiazhuang MOLBREEDING Biotechnology Co., Ltd.) and 2.7 μL of GenoBaits End Repair Enzyme were added into the tube, and water was replenished to 20 μL; the tube was put into an ABI 9700 PCR instrument for incubation for 20 minutes at 37° C. to complete the end repair and A addition process of the crushed fragments.

### 1.5 Sequencing Adapter Ligation of the Samples

The small tube was taken from the PCR instrument, 2 μL of GenoBaits Ultra DNA ligase, 8 μL of gGenoBaits Ultra DNA Ligase Buffer and 2 μL of GenoBaits Adapter were added into the tube, and water was replenished to 40 μL; then the tube was put on the ABI9700 PCR instrument for reaction for 30 minutes at 22° C. to complete the ligation of sequencing adapters.

### 1.6 DNA Purification of the Samples

48 μL of Beackman AMPure XP Beads (Beackman Company) was added to the ligation products for purification. After purification, magnetic beads were used for fragment screening, and the ligation products with inserted fragments of 200-300 bp were retained.

### 1.7 Library Amplification of the Samples

5 μL of a sequencing adapter with a Barcode sequence, 1 μL of a P5 adapter and 10 μL of GenoBaits PCR Master Mix were added into the PCR tube in the previous step, and pure water was replenished to 20 μL; amplification was carried out by ABI 9700 PCR instrument; the amplification procedures were: pre-denaturation at 95° C. for 5 minutes, denaturation at 95° C. for 30 seconds, annealing at 60° C. for 30 seconds, and extension at 72° C. for 30 seconds; denaturation, annealing and extension were repeated for a total of 8 cycles; extension at 72° C. for 5 min. Different Barcodes were used to distinguish different samples.

### 1.8 Library Purification of the Samples

24 μL of Beckmen AMPure XP Beads (Beackman Company) was added to the second round of the PCR product, which was then sucked up and down with a pipette for evenness; 0.2 μL of the PCR tube was placed on a magnetic rack until the solution was clear, the supernatant was discarded and the magnetic beads were washed once with 75 vol % ethanol; and the library DNA was eluted with Tris-HCl with pH value of 8.0.

2. The genotypes of the 894 SNP loci and 14 genes of the target samples were determined by the Low-density liquid-phase chip for Gossypium hirsutum L. based on targeted capture sequencing.

### 2.1 DNA Hybridization

500 ng of the constructed genomic DNA sequencing library was taken, into which 5 μL of GenoBaits Block I and 2 μL of GenoBaits Block II were added; it was then put on an Eppendorf Concentrator plus (Eppendorf Company) vacuum concentrator, and evaporated to dry powder at ≤70° C.; 8.5 μL of GenoBaits 2×Hyb Buffer, 2.7 μL of GenoBaits Hyb Buffer Enhancer and 2.8 μL of Nuclease-Free Water were added into the dry powder tube; the mixture was sucked and mixed evenly with a pipette, and then put on an ABI 9700 PCR instrument for incubation at 95° C. for 10 minutes; subsequently the PCR tube was taken out, and 3 μL of the synthesized probe (the concentration of the probe was 60 ng/μL) was added into the PCR tube; the mixture was subjected to vortex shaking to be mixed evenly; and then the PCR tube was put on ABI 9700 PCR instrument for incubation at 65° C. for 2 hours to complete the probe hybridization reaction.

### 2.2 DNA Capture

100 μL of GenoBaits DNA Probe Beads was added to the reaction system completed in the previous step, followed by sucking up and down for 10 times; the reaction system was then put in ABI 9700 PCR instrument for incubation at 65° C. for 45 minutes to bind the magnetic beads to the probe. 100 μL of GenoBaits Wash Buffer I and 150 μL of GenoBaits Wash BufferII were respectively used to hot-wash the magnetic beads bound with the probe at 65° C., and then 100 μL of GenoBaits Wash Buffer I, 150 μL of GenoBaits Wash Buffer II (and 150 μL of GenoBaits Wash Buffer III) were respectively used to wash the magnetic beads at room temperature. The washed magnetic beads were resuspended with 20 μL of Nuclease-Free Water.

13 μL of resuspended DNA (with magnetic beads) was added into a new 0.2 mL PCR tube, then 15 μL of GenoBaits PCR Master Mix and 2 μL of GenoBaits Primer Mix to configure a post-PCR system; the library was amplified with ABI 9700 PCR instrument. The amplification procedures were: pre-denaturation at 95° C. for 5 minutes, denaturation at 95° C. for 30 seconds, annealing at 60° C. for 30 seconds, and extension at 72° C. for 30 seconds; denaturation, annealing and extension were repeated for a total of 15 cycles; extension at 72° C. for 5 min.

45 μL of Beck Menam Pure XP Beads (Beackman Company) was added to the post-PCR product, followed by sucking up and down for evenness with a pipette; then the 0.2 mL PCR tube was placed on a magnetic rack until the solution was clear; the supernatant was discarded and the magnetic beads were washed twice with 75 vol % ethanol, and the library DNA were eluted with Tris-HCl with pH of 8.0. The hybridization capture of the probe was completed.

### 2.3 DNA Hybridization Capture Library Quality Inspection

The DNA concentration of the library was measured by Qubit Fluorometric Quantitation (Thermo Fisher), and then the fragment size of the library DNA was detected by agarose gel electrophoresis to determine whether it was between 300 to 400 bp.

### 2.4 DNA Hybridization Capture Library Sequencing

The constructed DNA library was sequenced by a Huada MGISEQ2000 sequencer.

### 2.5 Genotype Data Analysis

After quality data control of sequencing by FastQC (www.bioinformatics.babraham.ac.uk/project), the default parameters of BWA (bio-bwa.sourceforge.net) were used to compare the sequencing data to the reference genome. SNP identification for sequencing data were carried out by GATK (software.broadinstitute.org/gatk) software, and genotyping information of probe capture sequencing was extracted by self-compiled Perl script to form the final genotyping results, as shown in Table 2 and Table 3.

TABLE 2
Detection rate of low-density liquid-phase gene chip in 200 cotton varieties

| | | Number | Number | | | | |
|---|---|---|---|---|---|---|---|
| | | of | of | Detection | Total | | |
| | Locus | missing | detected | rate of | number | Number of | |
| No. | type | loci | loci | loci | of loci | sequences | Sample name |
| W4_23 | SNP | 31 | 863 | 0.965324 | 894 | 100249200 | Yu cotton 20 |
| W5_53 | SNP | 31 | 863 | 0.965324 | 894 | 99342600 | Y13 |
| W6_12 | SNP | 33 | 861 | 0.963087 | 894 | 99011100 | Shixuan 87*SGK321 |
| W6_20 | SNP | 33 | 861 | 0.963087 | 894 | 99988800 | 53--12 |
| W4_12 | SNP | 34 | 860 | 0.961969 | 894 | 99629100 | Xinluzao No. 25 |
| W6_2 | SNP | 35 | 859 | 0.96085 | 894 | 100976700 | Ken 62*HT23 |
| W7_41 | SNP | 35 | 859 | 0.96085 | 894 | 99422400 | 12-12-29 |
| W4_24 | SNP | 38 | 856 | 0.957494 | 894 | 100787400 | Xu cotton 18 |
| W7_51 | SNP | 38 | 856 | 0.957494 | 894 | 100119900 | 12-5-66 |
| W8_55 | SNP | 39 | 855 | 0.956376 | 894 | 99564300 | DP201 |
| W7_22 | SNP | 40 | 854 | 0.955257 | 894 | 100554900 | 25-27 |
| W4_26 | SNP | 41 | 853 | 0.954139 | 894 | 100237200 | Xinluzao No. 18 |
| W7_34 | SNP | 41 | 853 | 0.954139 | 894 | 100266000 | Dun cotton No. 1 |
| W7_52 | SNP | 41 | 853 | 0.954139 | 894 | 99038400 | 12-5-62 |
| W5_50 | SNP | 42 | 852 | 0.95302 | 894 | 100182300 | 111-43-50 |
| W5_56 | SNP | 42 | 852 | 0.95302 | 894 | 99903900 | Shi K14 |
| W6_45 | SNP | 42 | 852 | 0.95302 | 894 | 99362700 | Fuquan No. 10 |
| W6_57 | SNP | 42 | 852 | 0.95302 | 894 | 100907100 | Shikang 126 |
| W7_32 | SNP | 42 | 852 | 0.95302 | 894 | 100285200 | Jin 3 |
| W4_19 | SNP | 43 | 851 | 0.951902 | 894 | 100998300 | Yu cotton 112 |
| W6_47 | SNP | 43 | 851 | 0.951902 | 894 | 100189200 | Henan kanghuang |
| W7_43 | SNP | 43 | 851 | 0.951902 | 894 | 100931100 | 12-10-91 |
| W5_41 | SNP | 44 | 850 | 0.950783 | 894 | 99323400 | Nantong No. 5 |
| W6_14 | SNP | 44 | 850 | 0.950783 | 894 | 100806000 | 26-5*603 |
| W6_29 | SNP | 44 | 850 | 0.950783 | 894 | 99386400 | N78 |
| W6_38 | SNP | 44 | 850 | 0.950783 | 894 | 100534200 | ND012 |
| W7_55 | SNP | 44 | 850 | 0.950783 | 894 | 100782300 | 12-5-47 |
| W8_40 | SNP | 44 | 850 | 0.950783 | 894 | 99412800 | 15-23-52 |
| W8_53 | SNP | 44 | 850 | 0.950783 | 894 | 99927600 | 602 |
| W6_31 | SNP | 45 | 849 | 0.949664 | 894 | 99343500 | SW1 |
| W6_46 | SNP | 45 | 849 | 0.949664 | 894 | 100151700 | Haigan cotton |
| W8_21 | SNP | 45 | 849 | 0.949664 | 894 | 100318500 | 15-32-66 |
| W4_21 | SNP | 46 | 848 | 0.948546 | 894 | 100013100 | Xinluzao No. 35 |
| W5_57 | SNP | 46 | 848 | 0.948546 | 894 | 99984300 | 148-39 |
| W6_32 | SNP | 46 | 848 | 0.948546 | 894 | 100779900 | TH25 |
| W7_26 | SNP | 46 | 848 | 0.948546 | 894 | 100933800 | Han cotton 885 |
| W7_54 | SNP | 46 | 848 | 0.948546 | 894 | 99124200 | 12-5-50 |
| W8_29 | SNP | 46 | 848 | 0.948546 | 894 | 99807000 | 15-30-87 |
| W8_41 | SNP | 46 | 848 | 0.948546 | 894 | 100498500 | 15-23-2 |
| W4_10 | SNP | 47 | 847 | 0.947427 | 894 | 100618800 | Yu 79-10 |
| W4_11 | SNP | 47 | 847 | 0.947427 | 894 | 99957600 | Xinluzao No. 38 |
| W6_1 | SNP | 47 | 847 | 0.947427 | 894 | 99529800 | 19-34 |
| W6_33 | SNP | 47 | 847 | 0.947427 | 894 | 100663500 | Y17 |
| W6_52 | SNP | 47 | 847 | 0.947427 | 894 | 99811200 | Ji cotton 169 |
| W6_9 | SNP | 47 | 847 | 0.947427 | 894 | 99948900 | GK26* Xi 9 |
| W8_14 | SNP | 47 | 847 | 0.947427 | 894 | 99806400 | 15-49-128 |
| W8_3 | SNP | 47 | 847 | 0.947427 | 894 | 99324300 | 12-3-91 |
| W8_4 | SNP | 47 | 847 | 0.947427 | 894 | 100275300 | 12-3-79 |
| W4_15 | SNP | 48 | 846 | 0.946309 | 894 | 100711200 | Yan cotton No. 1 |
| W7_12 | SNP | 48 | 846 | 0.946309 | 894 | 99058800 | Huiyuan 14 |
| W7_35 | SNP | 48 | 846 | 0.946309 | 894 | 100970100 | Dezi cotton 531 |
| W7_49 | SNP | 48 | 846 | 0.946309 | 894 | 99326700 | 12-7-60 |
| W7_50 | SNP | 48 | 846 | 0.946309 | 894 | 99306900 | 12-7-59 |
| W7_7 | SNP | 48 | 846 | 0.946309 | 894 | 99368400 | Xinluzao No. 62 |
| W8_46 | SNP | 48 | 846 | 0.946309 | 894 | 100236300 | 15-22-119 |
| W4_20 | SNP | 49 | 845 | 0.94519 | 894 | 100209900 | Xuzhou 514 |
| W4_22 | SNP | 49 | 845 | 0.94519 | 894 | 100844100 | Qiannong 465 |
| W4_27 | SNP | 49 | 845 | 0.94519 | 894 | 100599600 | Xuzhou 58 |
| W5_49 | SNP | 49 | 845 | 0.94519 | 894 | 100093800 | 562*GK15 |
| W6_26 | SNP | 49 | 845 | 0.94519 | 894 | 100599900 | H65 |
| W6_56 | SNP | 49 | 845 | 0.94519 | 894 | 99428400 | Shi K10 |
| W7_17 | SNP | 49 | 845 | 0.94519 | 894 | 100983000 | K2725 |
| W7_18 | SNP | 49 | 845 | 0.94519 | 894 | 99957300 | Han 2301 |
| W7_25 | SNP | 49 | 845 | 0.94519 | 894 | 99270000 | Keke 1543 |
| W7_27 | SNP | 49 | 845 | 0.94519 | 894 | 99344400 | Kezi cotton 100 |
| W8_1 | SNP | 49 | 845 | 0.94519 | 894 | 99020100 | 12-4-4 |
| W8_9 | SNP | 49 | 845 | 0.94519 | 894 | 99031500 | 12-2-1 |
| W4_13 | SNP | 50 | 844 | 0.944072 | 894 | 99059400 | Lumianyan 32 |
| W4_18 | SNP | 50 | 844 | 0.944072 | 894 | 100920900 | Shu cotton No. 1 |
| W5_40 | SNP | 50 | 844 | 0.944072 | 894 | 99094200 | Zhongmiansuo 2 |
| W5_43 | SNP | 50 | 844 | 0.944072 | 894 | 99566100 | Su cotton No. 9 |
| W6_19 | SNP | 50 | 844 | 0.944072 | 894 | 99018000 | 9456D |
| W6_25 | SNP | 50 | 844 | 0.944072 | 894 | 99556800 | GK19 |
| W6_4 | SNP | 50 | 844 | 0.944072 | 894 | 99815400 | 9-8*41-79 |
| W6_5 | SNP | 50 | 844 | 0.944072 | 894 | 100613700 | 29-24 |
| W6_8 | SNP | 50 | 844 | 0.944072 | 894 | 99734700 | 2040048 |
| W7_13 | SNP | 50 | 844 | 0.944072 | 894 | 99374700 | Xinluzhong No. 42 |
| W7_2 | SNP | 50 | 844 | 0.944072 | 894 | 99099900 | Xinshixuan 12-2 |
| W7_31 | SNP | 50 | 844 | 0.944072 | 894 | 100951200 | Gan cotton 47 |
| W7_44 | SNP | 50 | 844 | 0.944072 | 894 | 100114500 | 12-10-55 |
| W8_28 | SNP | 50 | 844 | 0.944072 | 894 | 100653600 | 15-30-114 |
| W8_45 | SNP | 50 | 844 | 0.944072 | 894 | 100893600 | 15-22-122 |
| W5_51 | SNP | 51 | 843 | 0.942953 | 894 | 100363500 | Yahuang |
| W6_16 | SNP | 51 | 843 | 0.942953 | 894 | 99083400 | 86-27 |
| W6_23 | SNP | 51 | 843 | 0.942953 | 894 | 100297200 | FY408 |
| W7_59 | SNP | 51 | 843 | 0.942953 | 894 | 99893700 | 12-4-17 |
| W7_8 | SNP | 51 | 843 | 0.942953 | 894 | 100942800 | Hongye cotton |
| W8_15 | SNP | 51 | 843 | 0.942953 | 894 | 100924800 | 15-49-81 |
| W5_42 | SNP | 52 | 842 | 0.941834 | 894 | 100995300 | Jing 1246 |
| W6_21 | SNP | 52 | 842 | 0.941834 | 894 | 100983000 | Huiyuan 706 |
| W6_34 | SNP | 52 | 842 | 0.941834 | 894 | 99177600 | Xinshi H8 |
| W6_53 | SNP | 52 | 842 | 0.941834 | 894 | 99472200 | Jinhui No. 2 |
| W6_59 | SNP | 52 | 842 | 0.941834 | 894 | 99576000 | Xi 9 |
| W7_19 | SNP | 52 | 842 | 0.941834 | 894 | 99485100 | Zhongchuang 83 |
| W7_28 | SNP | 52 | 842 | 0.941834 | 894 | 99802500 | Guangyedaizi cotton |
| W7_29 | SNP | 52 | 842 | 0.941834 | 894 | 100461300 | Guannong No. 1 |
| W7_33 | SNP | 52 | 842 | 0.941834 | 894 | 100824600 | Ji cotton 25 |
| W7_37 | SNP | 52 | 842 | 0.941834 | 894 | 100739700 | Liao cotton No. 6 |
| W7_5 | SNP | 52 | 842 | 0.941834 | 894 | 100159500 | FY11 |
| W8_20 | SNP | 52 | 842 | 0.941834 | 894 | 100125300 | 15-34-37 |
| W8_27 | SNP | 52 | 842 | 0.941834 | 894 | 100520100 | 15-31-37 |
| W4_16 | SNP | 53 | 841 | 0.940716 | 894 | 99840300 | Xinluzao No. 37 |
| W4_25 | SNP | 53 | 841 | 0.940716 | 894 | 99889800 | Xinluzao No.34 |
| W5_46 | SNP | 53 | 841 | 0.940716 | 894 | 99389100 | 10-75(GK26) |
| W6_17 | SNP | 53 | 841 | 0.940716 | 894 | 99564000 | 44-32*0317 |
| W6_18 | SNP | 53 | 841 | 0.940716 | 894 | 100725000 | 907 |
| W6_30 | SNP | 53 | 841 | 0.940716 | 894 | 99855300 | Sic74 |
| W6_37 | SNP | 53 | 841 | 0.940716 | 894 | 99430800 | TH1246 |
| W7_16 | SNP | 53 | 841 | 0.940716 | 894 | 99008700 | Xinshi K25 |
| W7_21 | SNP | 53 | 841 | 0.940716 | 894 | 100089600 | Kelin 113 |
| W7_38 | SNP | 53 | 841 | 0.940716 | 894 | 99726900 | 9D208 |
| W8_23 | SNP | 53 | 841 | 0.940716 | 894 | 100993800 | 15-32-56 |
| W8_44 | SNP | 53 | 841 | 0.940716 | 894 | 100825200 | 15-22-123 |
| W5_44 | SNP | 54 | 840 | 0.939597 | 894 | 100837200 | 604 |
| W5_45 | SNP | 54 | 840 | 0.939597 | 894 | 100416000 | Shi K9*69-2 |
| W6_15 | SNP | 54 | 840 | 0.939597 | 894 | 100869000 | 78-12*27-67 |
| W7_58 | SNP | 54 | 840 | 0.939597 | 894 | 99997500 | 12-4-18 |
| W4_14 | SNP | 55 | 839 | 0.938479 | 894 | 100904400 | Yuwu 19 |
| W6_36 | SNP | 55 | 839 | 0.938479 | 894 | 100311900 | Z1112 |
| W7_20 | SNP | 55 | 839 | 0.938479 | 894 | 99243300 | Hexin 3-15 |
| W7_3 | SNP | 55 | 839 | 0.938479 | 894 | 100638300 | Yizao cotton No. 1 |
| W7_46 | SNP | 55 | 839 | 0.938479 | 894 | 99991800 | 12-7-63 |
| W8_16 | SNP | 55 | 839 | 0.938479 | 894 | 99406200 | 15-49-52 |
| W8_22 | SNP | 55 | 839 | 0.938479 | 894 | 99472200 | 15-32-58 |
| W8_35 | SNP | 55 | 839 | 0.938479 | 894 | 100480200 | 15-24-43 |
| W8_43 | SNP | 55 | 839 | 0.938479 | 894 | 99589500 | 15-22-138 |
| W8_51 | SNP | 55 | 839 | 0.938479 | 894 | 100058700 | 15-20-77 |
| W4_28 | SNP | 56 | 838 | 0.93736 | 894 | 99112500 | Xinqiu No. 4 |
| W6_11 | SNP | 56 | 838 | 0.93736 | 894 | 100515300 | 69--2 |
| W6_54 | SNP | 56 | 838 | 0.93736 | 894 | 100317000 | Liao cotton 18 |
| W7_14 | SNP | 56 | 838 | 0.93736 | 894 | 99093900 | B4-23 |
| W7_48 | SNP | 56 | 838 | 0.93736 | 894 | 99941700 | 12-7-61 |
| W8_8 | SNP | 56 | 838 | 0.93736 | 894 | 99772200 | 12-2-3 |
| W5_52 | SNP | 57 | 837 | 0.936242 | 894 | 100220400 | 116-39 Li |
| W5_55 | SNP | 57 | 837 | 0.936242 | 894 | 100485300 | Fengkai 2002 |
| W6_13 | SNP | 57 | 837 | 0.936242 | 894 | 99657600 | 69-2*68-38*44-5 |
| W6_55 | SNP | 57 | 837 | 0.936242 | 894 | 100733100 | Nongda 06-2 |
| W6_60 | SNP | 57 | 837 | 0.936242 | 894 | 99150900 | Xinken 09 |
| W8_12 | SNP | 57 | 837 | 0.936242 | 894 | 99462000 | 15-50-26 |
| W6_42 | SNP | 58 | 836 | 0.935123 | 894 | 100928700 | ZJ6 |
| W8_11 | SNP | 58 | 836 | 0.935123 | 894 | 100039800 | 12-1-41 |
| W8_32 | SNP | 58 | 836 | 0.935123 | 894 | 99022500 | 15-26-117 |
| W8_33 | SNP | 58 | 836 | 0.935123 | 894 | 99439800 | 15-26-115 |
| W5_47 | SNP | 59 | 835 | 0.934004 | 894 | 100508700 | Ken 4432*42-53 |
| W7_15 | SNP | 59 | 835 | 0.934004 | 894 | 99036900 | Dexia cotton No. 1 |
| W7_6 | SNP | 59 | 835 | 0.934004 | 894 | 100377900 | ZB0848 |
| W8_13 | SNP | 59 | 835 | 0.934004 | 894 | 100167300 | 15-50-24 |
| W8_6 | SNP | 59 | 835 | 0.934004 | 894 | 100583400 | 12-2-62 |
| W8_7 | SNP | 59 | 835 | 0.934004 | 894 | 99838800 | 12-2-43 |
| W5_54 | SNP | 60 | 834 | 0.932886 | 894 | 100389900 | 148-39*06X2 |
| W6_24 | SNP | 60 | 834 | 0.932886 | 894 | 100596600 | G2-2-5 |
| W6_3 | SNP | 60 | 834 | 0.932886 | 894 | 100425600 | (ZJ6*112-17)BC3 |
| W6_48 | SNP | 60 | 834 | 0.932886 | 894 | 99048000 | Huihe No. 28 ? |
| W6_7 | SNP | 60 | 834 | 0.932886 | 894 | 99922800 | 52-64*42-53K |
| W7_36 | SNP | 60 | 834 | 0.932886 | 894 | 100682400 | Ji cotton No. 1 |
| W7_42 | SNP | 60 | 834 | 0.932886 | 894 | 100202400 | 12-12-25 |
| W8_19 | SNP | 60 | 834 | 0.932886 | 894 | 100493400 | 15-34-38 |
| W6_28 | SNP | 61 | 833 | 0.931767 | 894 | 99491100 | JF-2 |
| W7_53 | SNP | 61 | 833 | 0.931767 | 894 | 99729900 | 12-5-61 |
| W5_58 | SNP | 62 | 832 | 0.930649 | 894 | 100466100 | 15-10 |
| W7_11 | SNP | 62 | 832 | 0.930649 | 894 | 100858500 | Huiyuan 12 |
| W7_47 | SNP | 62 | 832 | 0.930649 | 894 | 99605700 | 12-7-62 |
| W8_2 | SNP | 62 | 832 | 0.930649 | 894 | 99890100 | 12-3-100 |
| W8_34 | SNP | 62 | 832 | 0.930649 | 894 | 100190700 | 15-24-74 |
| W5_48 | SNP | 63 | 831 | 0.92953 | 894 | 99879600 | 41-79*Kang 10 |
| W6_40 | SNP | 63 | 831 | 0.92953 | 894 | 100251300 | C6-5-3-3 |
| W6_44 | SNP | 63 | 831 | 0.92953 | 894 | 100044000 | Bo 6 |
| W7_57 | SNP | 63 | 831 | 0.92953 | 894 | 100730100 | 12-4-65 |
| W8_10 | SNP | 63 | 831 | 0.92953 | 894 | 99693300 | 12-1-99 |
| W8_25 | SNP | 63 | 831 | 0.92953 | 894 | 99323700 | 15-31-127 |
| W8_5 | SNP | 63 | 831 | 0.92953 | 894 | 99361500 | 12-3-6 |
| W6_6 | SNP | 64 | 830 | 0.928412 | 894 | 99247500 | HY3(Yan) |
| W8_17 | SNP | 64 | 830 | 0.928412 | 894 | 99436200 | 15-48-27 |
| W8_26 | SNP | 64 | 830 | 0.928412 | 894 | 100524900 | 15-31-60 |
| W6_51 | SNP | 65 | 829 | 0.927293 | 894 | 100836000 | Huiyuan 720 |
| W7_10 | SNP | 65 | 829 | 0.927293 | 894 | 99849300 | Nongda cotton No. 8 |
| W7_60 | SNP | 65 | 829 | 0.927293 | 894 | 99119100 | 12-4-13 |
| W6_49 | SNP | 66 | 828 | 0.926174 | 894 | 100147800 | Xinluzao No. 61 |
| W8_24 | SNP | 66 | 828 | 0.926174 | 894 | 99160800 | 15-31-131 |
| W8_38 | SNP | 66 | 828 | 0.926174 | 894 | 99833100 | 15-23-71 |
| W8_54 | SNP | 66 | 828 | 0.926174 | 894 | 100014300 | 217 |
| W5_59 | SNP | 67 | 827 | 0.925056 | 894 | 100699200 | Huai cotton No. 2 |
| W6_10 | SNP | 67 | 827 | 0.925056 | 894 | 100195500 | C2 |
| W6_27 | SNP | 67 | 827 | 0.925056 | 894 | 99646800 | IF1-1K |
| W7_30 | SNP | 67 | 827 | 0.925056 | 894 | 99294000 | Jin cotton 29 |
| W6_22 | SNP | 68 | 826 | 0.923937 | 894 | 99210600 | Yan cotton 216 |
| W7_56 | SNP | 68 | 826 | 0.923937 | 894 | 99101400 | 12-4-94 |
| W8_52 | SNP | 68 | 826 | 0.923937 | 894 | 100813800 | Xinshi H 12 |
| W6_35 | SNP | 69 | 825 | 0.922819 | 894 | 100593900 | Tianyun 07195 |
| W6_43 | SNP | 69 | 825 | 0.922819 | 894 | 99564000 | Biaoda A parent |
| W8_30 | SNP | 70 | 824 | 0.9217 | 894 | 99070500 | 15-30-10 |
| W8_36 | SNP | 70 | 824 | 0.9217 | 894 | 99015300 | 15-24-42 |
| W7_4 | SNP | 71 | 823 | 0.920582 | 894 | 100414200 | Zhong 705 |
| W8_42 | SNP | 74 | 820 | 0.917226 | 894 | 99028800 | 15-23-1 |
| W7_9 | SNP | 75 | 819 | 0.916107 | 894 | 100838100 | 112-27 |
| W6_39 | SNP | 76 | 818 | 0.914989 | 894 | 99708300 | Jin ken 1242 |
| W8_37 | SNP | 78 | 816 | 0.912752 | 894 | 99621900 | 15-24-19 |
| W8_18 | SNP | 79 | 815 | 0.911633 | 894 | 99877200 | 15-37-106 |
| W8_50 | SNP | 80 | 814 | 0.910515 | 894 | 99403800 | 15-21-16 |
| W8_39 | SNP | 83 | 811 | 0.907159 | 894 | 99165900 | 15-23-68 |

TABLE 3
Detection of transgenic components in 200 cotton varieties by low-density
liquid-phase gene chip

| Gene | | | A3CP4- | A4CP4- | | | | A8cp4- | B. | B. | B. | B. | B. | |
|---|---|---|---|---|---|---|---|---|---|---|---|---|---|---|
| name | A1Bt | A2CPTI | epsps | epsps | A5bar | A6pat | A7CdP450 | epsps | 1CaMV35S | 2FMV35S | 3NOS | 4NOS | 5CaMV35S | NPTII |
| Num- | 64 | 0 | 0 | 3 | 0 | 0 | 0 | 0 | 68 | 3 | 0 | 54 | 0 | 64 |
| ber of | | | | | | | | | | | | | | |
| indi- | | | | | | | | | | | | | | |
| viduals | | | | | | | | | | | | | | |
| detected | | | | | | | | | | | | | | |
| W4_10 | 0 | 0 | 0 | 0 | 0 | 0 | 0 | 0 | 0 | 0 | 0 | 0 | 0 | 0 |
| W4_11 | 0 | 0 | 0 | 0 | 0 | 0 | 0 | 0 | 0 | 0 | 0 | 0 | 0 | 0 |
| W4_12 | 0 | 0 | 0 | 0 | 0 | 0 | 0 | 0 | 0 | 0 | 0 | 0 | 0 | 0 |
| W4_13 | 0 | 0 | 0 | 0 | 0 | 0 | 0 | 0 | 0 | 0 | 0 | 0 | 0 | 0 |
| W4_14 | 0 | 0 | 0 | 0 | 0 | 0 | 0 | 0 | 0 | 0 | 0 | 0 | 0 | 0 |
| W4_15 | 96.03% | 0 | 0 | 0 | 0 | 0 | 0 | 0 | 100.00% | 0 | 0 | 99.08% | 0 | 100.00% |
| W4_16 | 96.03% | 0 | 0 | 0 | 0 | 0 | 0 | 0 | 100.00% | 0 | 0 | 99.54% | 0 | 100.00% |
| W4_18 | 96.03% | 0 | 0 | 0 | 0 | 0 | 0 | 0 | 100.00% | 0 | 0 | 98.16% | 0 | 100.00% |
| W4_19 | 96.36% | 0 | 0 | 0 | 0 | 0 | 0 | 0 | 100.00% | 0 | 0 | 99.08% | 0 | 100.00% |
| W4_20 | 96.03% | 0 | 0 | 0 | 0 | 0 | 0 | 0 | 100.00% | 0 | 0 | 92.17% | 0 | 100.00% |
| W4_21 | 96.03% | 0 | 0 | 0 | 0 | 0 | 0 | 0 | 100.00% | 0 | 0 | 99.08% | 0 | 100.00% |
| W4_22 | 96.03% | 0 | 0 | 0 | 0 | 0 | 0 | 0 | 100.00% | 0 | 0 | 99.08% | 0 | 100.00% |
| W4_23 | 96.03% | 0 | 0 | 0 | 0 | 0 | 0 | 0 | 100.00% | 0 | 0 | 99.08% | 0 | 100.00% |
| W4_24 | 96.03% | 0 | 0 | 0 | 0 | 0 | 0 | 0 | 100.00% | 0 | 0 | 99.08% | 0 | 100.00% |
| W4_25 | 0 | 0 | 0 | 0 | 0 | 0 | 0 | 0 | 0 | 0 | 0 | 0 | 0 | 0 |
| W4_26 | 0 | 0 | 0 | 0 | 0 | 0 | 0 | 0 | 0 | 0 | 0 | 0 | 0 | 0 |
| W4_27 | 0 | 0 | 0 | 0 | 0 | 0 | 0 | 0 | 0 | 0 | 0 | 0 | 0 | 0 |
| W4_28 | 0 | 0 | 0 | 0 | 0 | 0 | 0 | 0 | 0 | 0 | 0 | 0 | 0 | 0 |
| W5_40 | 0 | 0 | 0 | 0 | 0 | 0 | 0 | 0 | 0 | 0 | 0 | 0 | 0 | 0 |
| W5_41 | 0 | 0 | 0 | 0 | 0 | 0 | 0 | 0 | 0 | 0 | 0 | 0 | 0 | 0 |
| W5_42 | 0 | 0 | 0 | 0 | 0 | 0 | 0 | 0 | 0 | 0 | 0 | 0 | 0 | 0 |
| W5_43 | 0 | 0 | 0 | 0 | 0 | 0 | 0 | 0 | 0 | 0 | 0 | 0 | 0 | 0 |
| W5_44 | 0 | 0 | 0 | 0 | 0 | 0 | 0 | 0 | 0 | 0 | 0 | 0 | 0 | 0 |
| W5_45 | 96.03% | 0 | 0 | 0 | 0 | 0 | 0 | 0 | 100.00% | 0 | 0 | 99.08% | 0 | 100.00% |
| W5_46 | 96.03% | 0 | 0 | 0 | 0 | 0 | 0 | 0 | 100.00% | 0 | 0 | 99.08% | 0 | 100.00% |
| W5_47 | 0 | 0 | 0 | 0 | 0 | 0 | 0 | 0 | 0 | 0 | 0 | 0 | 0 | 0 |
| W5_48 | 0 | 0 | 0 | 0 | 0 | 0 | 0 | 0 | 0 | 0 | 0 | 0 | 0 | 0 |
| W5_49 | 0 | 0 | 0 | 0 | 0 | 0 | 0 | 0 | 0 | 0 | 0 | 0 | 0 | 0 |
| W5_50 | 0 | 0 | 0 | 0 | 0 | 0 | 0 | 0 | 0 | 0 | 0 | 0 | 0 | 0 |
| W5_51 | 96.03% | 0 | 0 | 100.00% | 0 | 0 | 0 | 0 | 100.00% | 100.00% | 0 | 99.08% | 0 | 100.00% |
| W5_52 | 0 | 0 | 0 | 0 | 0 | 0 | 0 | 0 | 0 | 0 | 0 | 0 | 0 | 0 |
| W5_53 | 0 | 0 | 0 | 0 | 0 | 0 | 0 | 0 | 0 | 0 | 0 | 0 | 0 | 0 |
| W5_54 | 96.03% | 0 | 0 | 0 | 0 | 0 | 0 | 0 | 100.00% | 0 | 0 | 99.08% | 0 | 100.00% |
| W5_55 | 96.03% | 0 | 0 | 0 | 0 | 0 | 0 | 0 | 100.00% | 0 | 0 | 62.21% | 0 | 95.47% |
| W5_56 | 0 | 0 | 0 | 0 | 0 | 0 | 0 | 0 | 0 | 0 | 0 | 0 | 0 | 0 |
| W5_57 | 0 | 0 | 0 | 0 | 0 | 0 | 0 | 0 | 0 | 0 | 0 | 0 | 0 | 0 |
| W5_58 | 0 | 0 | 0 | 0 | 0 | 0 | 0 | 0 | 0 | 0 | 0 | 0 | 0 | 0 |
| W5_59 | 96.03% | 0 | 0 | 0 | 0 | 0 | 0 | 0 | 100.00% | 0 | 0 | 99.08% | 0 | 100.00% |
| W6_10 | 41.06% | 0 | 0 | 0 | 0 | 0 | 0 | 0 | 34.36% | 0 | 0 | 0 | 0 | 34.59% |
| W6_11 | 96.03% | 0 | 0 | 0 | 0 | 0 | 0 | 0 | 100.00% | 0 | 0 | 76.96% | 0 | 100.00% |
| W6_12 | 0 | 0 | 0 | 0 | 0 | 0 | 0 | 0 | 32.31% | 0 | 0 | 0 | 0 | 0 |
| W6_13 | 96.03% | 0 | 0 | 0 | 0 | 0 | 0 | 0 | 100.00% | 0 | 0 | 99.08% | 0 | 100.00% |
| W6_14 | 0 | 0 | 0 | 0 | 0 | 0 | 0 | 0 | 0 | 0 | 0 | 0 | 0 | 0 |
| W6_15 | 96.03% | 0 | 0 | 0 | 0 | 0 | 0 | 0 | 100.00% | 0 | 0 | 99.08% | 0 | 100.00% |
| W6_16 | 0 | 0 | 0 | 0 | 0 | 0 | 0 | 0 | 0 | 0 | 0 | 0 | 0 | 0 |
| W6_17 | 96.03% | 0 | 0 | 0 | 0 | 0 | 0 | 0 | 100.00% | 0 | 0 | 99.08% | 0 | 100.00% |
| W6_18 | 96.03% | 0 | 0 | 0 | 0 | 0 | 0 | 0 | 100.00% | 0 | 0 | 99.08% | 0 | 100.00% |
| W6_19 | 96.03% | 0 | 0 | 0 | 0 | 0 | 0 | 0 | 100.00% | 0 | 0 | 99.08% | 0 | 100.00% |
| W6_1 | 96.03% | 0 | 0 | 0 | 0 | 0 | 0 | 0 | 100.00% | 0 | 0 | 0 | 0 | 94.97% |
| W6_20 | 0 | 0 | 0 | 0 | 0 | 0 | 0 | 0 | 0 | 0 | 0 | 0 | 0 | 0 |
| W6_21 | 96.03% | 0 | 0 | 0 | 0 | 0 | 0 | 0 | 100.00% | 0 | 0 | 89.40% | 0 | 100.00% |
| W6_22 | 96.36% | 0 | 0 | 0 | 0 | 0 | 0 | 0 | 100.00% | 0 | 0 | 79.26% | 0 | 100.00% |
| W6_23 | 96.03% | 0 | 0 | 0 | 0 | 0 | 0 | 0 | 100.00% | 0 | 0 | 99.08% | 0 | 100.00% |
| W6_24 | 96.03% | 0 | 0 | 0 | 0 | 0 | 0 | 0 | 100.00% | 0 | 0 | 99.08% | 0 | 100.00% |
| W6_25 | 0 | 0 | 0 | 0 | 0 | 0 | 0 | 0 | 0 | 0 | 0 | 0 | 0 | 0 |
| W6_26 | 0 | 0 | 0 | 0 | 0 | 0 | 0 | 0 | 0 | 0 | 0 | 0 | 0 | 0 |
| W6_27 | 0 | 0 | 0 | 0 | 0 | 0 | 0 | 0 | 0 | 0 | 0 | 0 | 0 | 0 |
| W6_28 | 0 | 0 | 0 | 0 | 0 | 0 | 0 | 0 | 0 | 0 | 0 | 0 | 0 | 0 |
| W6_29 | 96.03% | 0 | 0 | 0 | 0 | 0 | 0 | 0 | 100.00% | 0 | 0 | 99.08% | 0 | 100.00% |
| W6_2 | 0 | 0 | 0 | 0 | 0 | 0 | 0 | 0 | 0 | 0 | 0 | 0 | 0 | 0 |
| W6_30 | 96.03% | 0 | 0 | 100.00% | 0 | 0 | 0 | 0 | 100.00% | 100.00% | 0 | 100.00% | 0 | 100.00% |
| W6_31 | 0 | 0 | 0 | 0 | 0 | 0 | 0 | 0 | 0 | 0 | 0 | 0 | 0 | 0 |
| W6_32 | 0 | 0 | 0 | 0 | 0 | 0 | 0 | 0 | 0 | 0 | 0 | 0 | 0 | 0 |
| W6_33 | 0 | 0 | 0 | 0 | 0 | 0 | 0 | 0 | 0 | 0 | 0 | 0 | 0 | 0 |
| W6_34 | 0 | 0 | 0 | 0 | 0 | 0 | 0 | 0 | 0 | 0 | 0 | 0 | 0 | 0 |
| W6_35 | 0 | 0 | 0 | 0 | 0 | 0 | 0 | 0 | 0 | 0 | 0 | 0 | 0 | 0 |
| W6_36 | 0 | 0 | 0 | 0 | 0 | 0 | 0 | 0 | 0 | 0 | 0 | 0 | 0 | 0 |
| W6_37 | 0 | 0 | 0 | 0 | 0 | 0 | 0 | 0 | 0 | 0 | 0 | 0 | 0 | 0 |
| W6_38 | 0 | 0 | 0 | 0 | 0 | 0 | 0 | 0 | 0 | 0 | 0 | 0 | 0 | 0 |
| W6_39 | 0 | 0 | 0 | 0 | 0 | 0 | 0 | 0 | 0 | 0 | 0 | 0 | 0 | 0 |
| W6_3 | 0 | 0 | 0 | 0 | 0 | 0 | 0 | 0 | 1.03% | 0 | 0 | 0 | 0 | 0 |
| W6_40 | 0 | 0 | 0 | 0 | 0 | 0 | 0 | 0 | 0 | 0 | 0 | 0 | 0 | 0 |
| W6_42 | 96.03% | 0 | 0 | 0 | 0 | 0 | 0 | 0 | 100.00% | 0 | 0 | 99.08% | 0 | 100.00% |
| W6_43 | 0 | 0 | 0 | 0 | 0 | 0 | 0 | 0 | 0 | 0 | 0 | 0 | 0 | 0 |
| W6_44 | 0 | 0 | 0 | 0 | 0 | 0 | 0 | 0 | 0 | 0 | 0 | 0 | 0 | 0 |
| W6_45 | 0 | 0 | 0 | 0 | 0 | 0 | 0 | 0 | 0 | 0 | 0 | 0 | 0 | 0 |
| W6_46 | 96.03% | 0 | 0 | 100.00% | 0 | 0 | 0 | 0 | 100.00% | 100.00% | 0 | 99.54% | 0 | 100.00% |
| W6_47 | 0 | 0 | 0 | 0 | 0 | 0 | 0 | 0 | 0 | 0 | 0 | 0 | 0 | 0 |
| W6_48 | 0 | 0 | 0 | 0 | 0 | 0 | 0 | 0 | 0 | 0 | 0 | 0 | 0 | 0 |
| W6_49 | 0 | 0 | 0 | 0 | 0 | 0 | 0 | 0 | 0 | 0 | 0 | 0 | 0 | 0 |
| W6_4 | 96.03% | 0 | 0 | 0 | 0 | 0 | 0 | 0 | 100.00% | 0 | 0 | 96.31% | 0 | 100.00% |
| W6_51 | 96.03% | 0 | 0 | 0 | 0 | 0 | 0 | 0 | 100.00% | 0 | 0 | 70.97% | 0 | 100.00% |
| W6_52 | 0 | 0 | 0 | 0 | 0 | 0 | 0 | 0 | 0 | 0 | 0 | 0 | 0 | 0 |
| W6_53 | 0 | 0 | 0 | 0 | 0 | 0 | 0 | 0 | 0 | 0 | 0 | 0 | 0 | 0 |
| W6_54 | 96.03% | 0 | 0 | 0 | 0 | 0 | 0 | 0 | 100.00% | 0 | 0 | 99.08% | 0 | 100.00% |
| W6_55 | 0 | 0 | 0 | 0 | 0 | 0 | 0 | 0 | 0 | 0 | 0 | 0 | 0 | 0 |
| W6_56 | 0 | 0 | 0 | 0 | 0 | 0 | 0 | 0 | 44.10% | 0 | 0 | 0 | 0 | 0 |
| W6_57 | 96.03% | 0 | 0 | 0 | 0 | 0 | 0 | 0 | 100.00% | 0 | 0 | 99.08% | 0 | 100.00% |
| W6_59 | 0 | 0 | 0 | 0 | 0 | 0 | 0 | 0 | 0 | 0 | 0 | 0 | 0 | 0 |
| W6_5 | 0 | 0 | 0 | 0 | 0 | 0 | 0 | 0 | 0 | 0 | 0 | 0 | 0 | 0 |
| W6_60 | 96.03% | 0 | 0 | 0 | 0 | 0 | 0 | 0 | 100.00% | 0 | 0 | 99.08% | 0 | 100.00% |
| W6_6 | 0 | 0 | 0 | 0 | 0 | 0 | 0 | 0 | 15.90% | 0 | 0 | 0 | 0 | 0 |
| W6_7 | 96.03% | 0 | 0 | 0 | 0 | 0 | 0 | 0 | 100.00% | 0 | 0 | 44.24% | 0 | 88.30% |
| W6_8 | 5.63% | 0 | 0 | 0 | 0 | 0 | 0 | 0 | 62.56% | 0 | 0 | 0 | 0 | 8.05% |
| W6_9 | 96.03% | 0 | 0 | 0 | 0 | 0 | 0 | 0 | 100.00% | 0 | 0 | 99.08% | 0 | 100.00% |
| W7_10 | 96.03% | 0 | 0 | 0 | 0 | 0 | 0 | 0 | 100.00% | 0 | 0 | 99.08% | 0 | 100.00% |
| W7_11 | 96.03% | 0 | 0 | 0 | 0 | 0 | 0 | 0 | 100.00% | 0 | 0 | 55.30% | 0 | 99.25% |
| W7_12 | 11.26% | 0 | 0 | 0 | 0 | 0 | 0 | 0 | 0 | 0 | 0 | 0 | 0 | 0 |
| W7_13 | 31.13% | 0 | 0 | 0 | 0 | 0 | 0 | 0 | 63.59% | 0 | 0 | 0 | 0 | 0 |
| W7_14 | 0 | 0 | 0 | 0 | 0 | 0 | 0 | 0 | 0 | 0 | 0 | 0 | 0 | 0 |
| W7_15 | 0 | 0 | 0 | 0 | 0 | 0 | 0 | 0 | 0 | 0 | 0 | 0 | 0 | 0 |
| W7_16 | 96.03% | 0 | 0 | 0 | 0 | 0 | 0 | 0 | 100.00% | 0 | 0 | 99.08% | 0 | 100.00% |
| W7_17 | 86.75% | 0 | 0 | 0 | 0 | 0 | 0 | 0 | 75.38% | 0 | 0 | 0 | 0 | 27.42% |
| W7_18 | 96.03% | 0 | 0 | 0 | 0 | 0 | 0 | 0 | 100.00% | 0 | 0 | 99.08% | 0 | 100.00% |
| W7_19 | 0 | 0 | 0 | 0 | 0 | 0 | 0 | 0 | 0 | 0 | 0 | 0 | 0 | 0 |
| W7_20 | 71.19% | 0 | 0 | 0 | 0 | 0 | 0 | 0 | 99.49% | 0 | 0 | 34.10% | 0 | 45.53% |
| W7_21 | 96.03% | 0 | 0 | 0 | 0 | 0 | 0 | 0 | 100.00% | 0 | 0 | 82.03% | 0 | 100.00% |
| W7_22 | 0 | 0 | 0 | 0 | 0 | 0 | 0 | 0 | 0 | 0 | 0 | 0 | 0 | 0 |
| W7_25 | 96.03% | 0 | 0 | 0 | 0 | 0 | 0 | 0 | 100.00% | 0 | 0 | 99.08% | 0 | 100.00% |
| W7_26 | 96.03% | 0 | 0 | 0 | 0 | 0 | 0 | 0 | 100.00% | 0 | 0 | 99.08% | 0 | 100.00% |
| W7_27 | 0 | 0 | 0 | 0 | 0 | 0 | 0 | 0 | 0 | 0 | 0 | 0 | 0 | 0 |
| W7_28 | 0 | 0 | 0 | 0 | 0 | 0 | 0 | 0 | 0 | 0 | 0 | 0 | 0 | 0 |
| W7_29 | 0 | 0 | 0 | 0 | 0 | 0 | 0 | 0 | 0 | 0 | 0 | 0 | 0 | 0 |
| W7_2 | 0 | 0 | 0 | 0 | 0 | 0 | 0 | 0 | 0 | 0 | 0 | 0 | 0 | 0 |
| W7_30 | 0 | 0 | 0 | 0 | 0 | 0 | 0 | 0 | 0 | 0 | 0 | 0 | 0 | 0 |
| W7_31 | 0 | 0 | 0 | 0 | 0 | 0 | 0 | 0 | 0 | 0 | 0 | 0 | 0 | 0 |
| W7_32 | 0 | 0 | 0 | 0 | 0 | 0 | 0 | 0 | 0 | 0 | 0 | 0 | 0 | 0 |
| W7_33 | 0 | 0 | 0 | 0 | 0 | 0 | 0 | 0 | 0 | 0 | 0 | 0 | 0 | 0 |
| W7_34 | 0 | 0 | 0 | 0 | 0 | 0 | 0 | 0 | 0 | 0 | 0 | 0 | 0 | 0 |
| W7_35 | 96.03% | 0 | 0 | 0 | 0 | 0 | 0 | 0 | 100.00% | 0 | 0 | 99.08% | 0 | 100.00% |
| W7_36 | 96.03% | 0 | 0 | 0 | 0 | 0 | 0 | 0 | 100.00% | 0 | 0 | 61.29% | 0 | 100.00% |
| W7_37 | 96.03% | 0 | 0 | 0 | 0 | 0 | 0 | 0 | 100.00% | 0 | 0 | 99.08% | 0 | 95.85% |
| W7_38 | 0 | 0 | 0 | 0 | 0 | 0 | 0 | 0 | 0 | 0 | 0 | 0 | 0 | 0 |
| W7_3 | 0 | 0 | 0 | 0 | 0 | 0 | 0 | 0 | 0 | 0 | 0 | 0 | 0 | 0 |
| W7_41 | 94.04% | 0 | 0 | 0 | 0 | 0 | 0 | 0 | 70.77% | 0 | 0 | 33.18% | 0 | 28.05% |
| W7_42 | 0 | 0 | 0 | 0 | 0 | 0 | 0 | 0 | 0 | 0 | 0 | 0 | 0 | 0 |
| W7_43 | 0 | 0 | 0 | 0 | 0 | 0 | 0 | 0 | 0 | 0 | 0 | 0 | 0 | 0 |
| W7_44 | 0 | 0 | 0 | 0 | 0 | 0 | 0 | 0 | 0 | 0 | 0 | 0 | 0 | 0 |
| W7_46 | 0 | 0 | 0 | 0 | 0 | 0 | 0 | 0 | 0 | 0 | 0 | 0 | 0 | 0 |
| W7_47 | 0 | 0 | 0 | 0 | 0 | 0 | 0 | 0 | 0 | 0 | 0 | 0 | 0 | 0 |
| W7_48 | 0 | 0 | 0 | 0 | 0 | 0 | 0 | 0 | 0 | 0 | 0 | 0 | 0 | 0 |
| W7_49 | 0 | 0 | 0 | 0 | 0 | 0 | 0 | 0 | 0 | 0 | 0 | 0 | 0 | 0 |
| W7_4 | 96.03% | 0 | 0 | 0 | 0 | 0 | 0 | 0 | 100.00% | 0 | 0 | 83.41% | 0 | 98.87% |
| W7_50 | 0 | 0 | 0 | 0 | 0 | 0 | 0 | 0 | 0 | 0 | 0 | 0 | 0 | 0 |
| W7_51 | 0 | 0 | 0 | 0 | 0 | 0 | 0 | 0 | 0 | 0 | 0 | 0 | 0 | 0 |
| W7_52 | 0 | 0 | 0 | 0 | 0 | 0 | 0 | 0 | 0 | 0 | 0 | 0 | 0 | 0 |
| W7_53 | 0 | 0 | 0 | 0 | 0 | 0 | 0 | 0 | 0 | 0 | 0 | 0 | 0 | 0 |
| W7_54 | 0 | 0 | 0 | 0 | 0 | 0 | 0 | 0 | 0 | 0 | 0 | 0 | 0 | 0 |
| W7_55 | 0 | 0 | 0 | 0 | 0 | 0 | 0 | 0 | 0 | 0 | 0 | 0 | 0 | 0 |
| W7_56 | 0 | 0 | 0 | 0 | 0 | 0 | 0 | 0 | 0 | 0 | 0 | 0 | 0 | 0 |
| W7_57 | 0 | 0 | 0 | 0 | 0 | 0 | 0 | 0 | 0 | 0 | 0 | 0 | 0 | 0 |
| W7_58 | 0 | 0 | 0 | 0 | 0 | 0 | 0 | 0 | 0 | 0 | 0 | 0 | 0 | 0 |
| W7_59 | 0 | 0 | 0 | 0 | 0 | 0 | 0 | 0 | 0 | 0 | 0 | 0 | 0 | 0 |
| W7_5 | 0 | 0 | 0 | 0 | 0 | 0 | 0 | 0 | 10.26% | 0 | 0 | 0 | 0 | 0 |
| W7_60 | 0 | 0 | 0 | 0 | 0 | 0 | 0 | 0 | 0 | 0 | 0 | 0 | 0 | 0 |
| W7_6 | 96.03% | 0 | 0 | 0 | 0 | 0 | 0 | 0 | 100.00% | 0 | 0 | 99.08% | 0 | 100.00% |
| W7_7 | 96.03% | 0 | 0 | 0 | 0 | 0 | 0 | 0 | 100.00% | 0 | 0 | 99.08% | 0 | 100.00% |
| W7_8 | 0 | 0 | 0 | 0 | 0 | 0 | 0 | 0 | 0 | 0 | 0 | 0 | 0 | 0 |
| W7_9 | 96.03% | 0 | 0 | 0 | 0 | 0 | 0 | 0 | 100.00% | 0 | 0 | 0 | 0 | 83.90% |
| W8_10 | 0 | 0 | 0 | 0 | 0 | 0 | 0 | 0 | 0 | 0 | 0 | 0 | 0 | 0 |
| W8_11 | 0 | 0 | 0 | 0 | 0 | 0 | 0 | 0 | 0 | 0 | 0 | 0 | 0 | 0 |
| W8_12 | 0 | 0 | 0 | 0 | 0 | 0 | 0 | 0 | 0 | 0 | 0 | 0 | 0 | 0 |
| W8_13 | 0 | 0 | 0 | 0 | 0 | 0 | 0 | 0 | 0 | 0 | 0 | 0 | 0 | 0 |
| W8_14 | 0 | 0 | 0 | 0 | 0 | 0 | 0 | 0 | 0 | 0 | 0 | 0 | 0 | 0 |
| W8_15 | 0 | 0 | 0 | 0 | 0 | 0 | 0 | 0 | 0 | 0 | 0 | 0 | 0 | 0 |
| W8_16 | 0 | 0 | 0 | 0 | 0 | 0 | 0 | 0 | 0 | 0 | 0 | 0 | 0 | 0 |
| W8_17 | 0 | 0 | 0 | 0 | 0 | 0 | 0 | 0 | 0 | 0 | 0 | 0 | 0 | 0 |
| W8_18 | 0 | 0 | 0 | 0 | 0 | 0 | 0 | 0 | 0 | 0 | 0 | 0 | 0 | 0 |
| W8_19 | 0 | 0 | 0 | 0 | 0 | 0 | 0 | 0 | 0 | 0 | 0 | 0 | 0 | 0 |
| W8_1 | 0 | 0 | 0 | 0 | 0 | 0 | 0 | 0 | 0 | 0 | 0 | 0 | 0 | 0 |
| W8_20 | 0 | 0 | 0 | 0 | 0 | 0 | 0 | 0 | 0 | 0 | 0 | 0 | 0 | 0 |
| W8_21 | 0 | 0 | 0 | 0 | 0 | 0 | 0 | 0 | 0 | 0 | 0 | 0 | 0 | 0 |
| W8_22 | 0 | 0 | 0 | 0 | 0 | 0 | 0 | 0 | 0 | 0 | 0 | 0 | 0 | 0 |
| W8_23 | 0 | 0 | 0 | 0 | 0 | 0 | 0 | 0 | 0 | 0 | 0 | 0 | 0 | 0 |
| W8_24 | 0 | 0 | 0 | 0 | 0 | 0 | 0 | 0 | 0 | 0 | 0 | 0 | 0 | 0 |
| W8_25 | 0 | 0 | 0 | 0 | 0 | 0 | 0 | 0 | 0 | 0 | 0 | 0 | 0 | 0 |
| W8_26 | 0 | 0 | 0 | 0 | 0 | 0 | 0 | 0 | 0 | 0 | 0 | 0 | 0 | 0 |
| W8_27 | 0 | 0 | 0 | 0 | 0 | 0 | 0 | 0 | 0 | 0 | 0 | 0 | 0 | 0 |
| W8_28 | 0 | 0 | 0 | 0 | 0 | 0 | 0 | 0 | 0 | 0 | 0 | 0 | 0 | 0 |
| W8_29 | 0 | 0 | 0 | 0 | 0 | 0 | 0 | 0 | 0 | 0 | 0 | 0 | 0 | 0 |
| W8_2 | 0 | 0 | 0 | 0 | 0 | 0 | 0 | 0 | 0 | 0 | 0 | 0 | 0 | 0 |
| W8_30 | 28.15% | 0 | 0 | 0 | 0 | 0 | 0 | 0 | 0 | 0 | 0 | 0 | 0 | 4.15% |
| W8_32 | 0 | 0 | 0 | 0 | 0 | 0 | 0 | 0 | 0 | 0 | 0 | 0 | 0 | 0 |
| W8_33 | 0 | 0 | 0 | 0 | 0 | 0 | 0 | 0 | 0 | 0 | 0 | 0 | 0 | 0 |
| W8_34 | 0 | 0 | 0 | 0 | 0 | 0 | 0 | 0 | 49.23% | 0 | 0 | 0 | 0 | 10.57% |
| W8_35 | 0 | 0 | 0 | 0 | 0 | 0 | 0 | 0 | 0 | 0 | 0 | 0 | 0 | 0 |
| W8_36 | 96.03% | 0 | 0 | 0 | 0 | 0 | 0 | 0 | 100.00% | 0 | 0 | 0 | 0 | 89.18% |
| W8_37 | 0 | 0 | 0 | 0 | 0 | 0 | 0 | 0 | 0 | 0 | 0 | 0 | 0 | 0 |
| W8_38 | 96.03% | 0 | 0 | 0 | 0 | 0 | 0 | 0 | 100.00% | 0 | 0 | 99.08% | 0 | 100.00% |
| W8_39 | 96.03% | 0 | 0 | 0 | 0 | 0 | 0 | 0 | 100.00% | 0 | 0 | 79.26% | 0 | 100.00% |
| W8_3 | 0 | 0 | 0 | 0 | 0 | 0 | 0 | 0 | 0 | 0 | 0 | 0 | 0 | 0 |
| W8_40 | 96.36% | 0 | 0 | 0 | 0 | 0 | 0 | 0 | 100.00% | 0 | 0 | 99.08% | 0 | 100.00% |
| W8_41 | 0 | 0 | 0 | 0 | 0 | 0 | 0 | 0 | 0 | 0 | 0 | 0 | 0 | 0 |
| W8_42 | 0 | 0 | 0 | 0 | 0 | 0 | 0 | 0 | 0 | 0 | 0 | 0 | 0 | 0 |
| W8_43 | 96.03% | 0 | 0 | 0 | 0 | 0 | 0 | 0 | 100.00% | 0 | 0 | 0 | 0 | 89.81% |
| W8_44 | 0 | 0 | 0 | 0 | 0 | 0 | 0 | 0 | 0 | 0 | 0 | 0 | 0 | 0 |
| W8_45 | 0 | 0 | 0 | 0 | 0 | 0 | 0 | 0 | 0 | 0 | 0 | 0 | 0 | 0 |
| W8_46 | 0 | 0 | 0 | 0 | 0 | 0 | 0 | 0 | 0 | 0 | 0 | 0 | 0 | 0 |
| W8_4 | 0 | 0 | 0 | 0 | 0 | 0 | 0 | 0 | 0 | 0 | 0 | 0 | 0 | 0 |
| W8_50 | 0 | 0 | 0 | 0 | 0 | 0 | 0 | 0 | 0 | 0 | 0 | 0 | 0 | 7.30% |
| W8_51 | 0 | 0 | 0 | 0 | 0 | 0 | 0 | 0 | 0 | 0 | 0 | 0 | 0 | 0 |
| W8_52 | 0 | 0 | 0 | 0 | 0 | 0 | 0 | 0 | 0 | 0 | 0 | 0 | 0 | 0 |
| W8_53 | 0 | 0 | 0 | 0 | 0 | 0 | 0 | 0 | 0 | 0 | 0 | 0 | 0 | 0 |
| W8_54 | 0 | 0 | 0 | 0 | 0 | 0 | 0 | 0 | 0 | 0 | 0 | 0 | 0 | 0 |
| W8_55 | 0 | 0 | 0 | 0 | 0 | 0 | 0 | 0 | 0 | 0 | 0 | 0 | 0 | 0 |
| W8_5 | 0 | 0 | 0 | 0 | 0 | 0 | 0 | 0 | 0 | 0 | 0 | 0 | 0 | 0 |
| W8_6 | 0 | 0 | 0 | 0 | 0 | 0 | 0 | 0 | 0 | 0 | 0 | 0 | 0 | 0 |
| W8_7 | 0 | 0 | 0 | 0 | 0 | 0 | 0 | 0 | 0 | 0 | 0 | 0 | 0 | 0 |
| W8_8 | 0 | 0 | 0 | 0 | 0 | 0 | 0 | 0 | 0 | 0 | 0 | 0 | 0 | 0 |
| W8_9 | 0 | 0 | 0 | 0 | 0 | 0 | 0 | 0 | 0 | 0 | 0 | 0 | 0 | 0 |

The above results confirmed that the detection rate of low-density SNP liquid-phase chip in 200 Gossypium hirsutum L. materials was 90.7%-96.5%, and the average locus detection rate was 94%. It has high polymorphism in Gossypium hirsutum L. varieties, which is very suitable for Gossypium hirsutum L. germplasm resource evaluation, genetic improvement and transgenic component detection with high detection accuracy. Compared with existing solid-phase chips and developed high-density liquid-phase chips, cotton breeding research can be carried out at lower costs.

Obviously, the above examples are only intended for clear explanation, rather than a limitation on the implementation. For those skilled in the art, other variations or changes in different forms can be made on the basis of the above description. It is not necessary and impossible to exhaust all the embodiments here. Nevertheless, obvious changes or variations derived therefrom still fall within the scope of protection of the present disclosure.

What is claimed is:

1. A low-density liquid-phase chip for Gossypium hirsutum L. based on targeted capture sequencing, wherein a whole genome low-density SNP chip of Gossypium hirsutum L. consists of probes for detecting 894 SNP loci and 14 major transgenes; the 14 major transgenes are specifically: A1Bt, A2CPTI, A3CP4-epsps, A4CP4-epsps, A5bar, A6pat, A7CdP450, A8cp4-epsps, B.1CaMV35S, B.2FMV35S, B.3NOS, B.4NOS, B.5CaMV35S and NPTII; and the 894 SNP loci are shown in Table 1.

2. The low-density liquid-phase chip for Gossypium hirsutum L. based on targeted capture sequencing according to claim 1, wherein the probes for detecting the 14 major transgenes are shown as SEQ ID NOs. 1-14.

3. Use of the low-density liquid-phase chip for Gossypium hirsutum L. based on targeted capture sequencing according to claim 1, comprising detection and genotyping of transgenic components of Gossypium hirsutum L., resource evaluation and lineage identification of Gossypium hirsutum L., seed purity identification, and genetic improvement of major agronomic traits of Gossypium hirsutum L. varieties.

4. The use according to claim 3, wherein the major agronomic traits comprise fiber quality trait, yield trait, and disease resistance.


SEQUENCE LISTING (14 sequences)

SEQ ID NO: 1 - DNA, 302 bp, synthetic construct
  source 1..302: mol_type = other DNA
gaaggtttctagcaatctctaccaaatctatgcagagagcttcagagagtgggaagccgatcctactaacccagctctccgcgaggaaatgcgtattcaattcaacgacatgaacagcgccttgaccacagctatcccattgttcgcagtccagaactaccaagttcctctcttgtccgtgtacgttcaagcagctaatcttcacctcagcgtgcttcgagacgttagcgtgtttgggcaaaggtggggattcgatgctgcaaccatcaatagccgttacaacgaccttactaggctgatcg

SEQ ID NO: 2 - DNA, 243 bp, synthetic construct
  source 1..243: mol_type = other DNA
gatctgaaccacctcggaagtaatcatcatgatgactcaagcgatgaaccttctgagtcttcagaaccatgctgcgattcatgcatctgcactaaatcaatacctcctcaatgccattgtacagatatcaggttgaattcgtgtcactcggcttgcaaatcctgcatgtgtacacgatcaatgccaggcaagtgtcgttgccttgacattgctgatttctgttacaaaccttgcaagtccagg

SEQ ID NO: 3 - DNA, 333 bp, synthetic construct
  source 1..333: mol_type = other DNA
  modified_base 14: mod_base = i
acggtgaccgtcttcccgttaccntgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttc

SEQ ID NO: 4 - DNA, 333 bp, synthetic construct
  source 1..333: mol_type = other DNA
acggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttc

SEQ ID NO: 5 - DNA, 262 bp, synthetic construct
  source 1..262: mol_type = other DNA
gaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctgg

SEQ ID NO: 6 - DNA, 263 bp, synthetic construct
  source 1..263: mol_type = other DNA
gaaggctaggaacgcttacgattcgacagttgagagtactgtttacgtgtcacataggcatcaaaggttgggcctaggatccacattgtacacacatttgcttaagtctatggaggcgcaaggttttaagtctgtggttgctgttatagggccttccaaacgatccatctgttaggttgcatgaggctttgggatacacagcccggggtacattgcgcgcagctggatacaagcatggtggatggcatgatgttggtttttgg

SEQ ID NO: 7 - DNA, 1551 bp, synthetic construct
  source 1..1551: mol_type = other DNA
atggataaggcctacgtggccctcctctccttcgcctccctcttcttgctccactacctcgtttcccgccgcaatggcaccgggaagggcagcaaggccaagggcgcgctgccgccaagccctccatccgttccgttcctgggccacctccaccttgtcaagacgccattccacgctgcgctggcacgcctcgcggactgccacggcccggtcttctccctgcggatgggagcccgccccgcagttgtggtgtcctcgccggagcacgccaaggagtgcttcacggagcacgacgtggccttcgccaaccggccgcgctttccctcgcagcagctcgcctccttcaacggtgccgcgctgggttccgccagctacggcccgtactggcgcaacctccgccgcgtcgccaccgtccacctcctgtccgcgcaccgcgtcgcgtgcatgacggggactatcgcggccgaggtgcgggccatggtgcgacggatgaaccgcgccgcgcaggtggcatcaggcggcgcggcgcgcatcgagctcaagcggaggctatttgaggtctcgctcagcgtgcttatggagaccatcgcgcggaccaagacgtcacgtacggaggcggacgacgacacggacatgtcgcctgaggcccgggagttcaagcagatcgtggatgagctcctgcctcacctcggcacggctaacttgtgggactacatgccggtgttgcggtggttcgacgtgttcggcgtgaggaagaagatcgtgtccgcggtgaggagaagggacgcgttcctgcggcatcttgtcgacgcagagaggacgaggctggacgacggcaacgatgcgggcgagaagaagagcatcattgctatgctgctcactctgcagaagtcagagccggacgtctactcggataccatgatcatggctctatgtgggaacttgtttggggccggcacagagaccacgtcgacgaccaccgaatgggccatgtctctcctcctcaaccacccggagaagctcaggaaggcgcaggctgagatcgatgctgtcgtgggcacatcccgccttcttaccgccgacgacatgcctcgtctcacctacctccgctgcatcatcgacgagaccatgcgcctgtacccggccgcaccacttctgctgccacacgagtcctcgacacactgcaaggtcggcggctacgacgtgcccgccggcacgatgctgctcgtcaacgtgtacgccatccacagggaccccgcggtgtgggacgggccgaccgagttcgtgccggagcggttcgaggatggcaaggcagaaggccggctgctgatgccgttcgggatgggacggcgcaagtgtcccggcgagacgctcgcgctgcggacgatcgggctggtgctcggcacgctgatccagtgtttcgactgggaccgggttgatggtcttgaggtcgacatgactgaaagtggtgggctcacgatccccagggctgtcccgttggaggccatgtgcaggcctcgtgcgacgatgcgtgaggttttgcaggagctc

SEQ ID NO: 8 - DNA, 1590 bp, synthetic construct
  source 1..1590: mol_type = other DNA
atggcggcgaccatggcgtccaacgctgcggctgcggctgcggtgtccctggaccaggccgtggctgcgtcggcagcgttctcgtcgcggaagcagctgcggctgcctgccgcagcgcgcggagggatgcgggtgcgggtgcgggcgcggggtcggcgggaggcggtggtggtggcgtccgcgtcgtcgtcgtcggtggcagcgccggcggcgaaggctgagatgctacacggtgcaagcagccggccggcaaccgctcgcaaatcttccggcctttcgggaacggtcaggattccgggcgataagtccatatcccaccggtcgttcatgttcggcggtcttgccagcggtgagacgcgcatcacgggcctgcttgaaggtgaggacgtgatcaataccgggaaggccatgcaggctatgggagcgcgtatccgcaaggaaggtgacacatggatcattgacggcgttgggaatggcggtctgctcgcccctgaggcccctctcgacttcggcaatgcggcgacgggctgcaggctcactatgggactggtcggggtgtacgacttcgatagcacgttcatcggagacgcctcgctcacaaagcgcccaatgggccgcgttctgaacccgttgcgcgagatgggcgtacaggtcaaatccgaggatggtgaccgtttgcccgttacgctgcgcgggccgaagacgcctaccccgattacctaccgcgtgccaatggcatccgcccaggtcaagtcagccgtgctcctcgccggactgaacactccgggcatcaccacggtgatcgagcccatcatgaccagggatcataccgaaaagatgcttcaggggtttggcgccaacctgacggtcgagacggacgctgacggcgtcaggaccatccgccttgagggcaggggtaaactgactggccaagtcatcgatgttccgggagacccgtcgtccacggccttcccgttggttgcggcgctgctcgtgccggggagtgacgtgaccatcctgaacgtcctcatgaacccgaccaggaccggcctgatcctcacgcttcaggagatgggagccgacatcgaggtgatcaacccgcgcctggcaggcggtgaagacgttgcggatctgcgcgtgcgctcctctaccctgaagggcgtgacggtcccggaagatcgcgcgccgtccatgatagacgagtatcctattctggccgtcgccgctgcgttcgccgaaggggccacggtcatgaacggtcttgaggaactccgcgtgaaggaatcggatcgcctgtcggcggtggccaatggcctgaagctcaacggtgttgactgcgacgagggtgagacctcactcgtggtccgtggccggcctgatggcaagggcctcggcaacgccagtggagcggccgtcgccacgcacctcgatcatcgcatcgcgatgtccttcttggtgatgggtctcgtctcagagaacccggtgaccgtcgatgacgccacgatgatagcgacgagcttcccagagttcatggatctgatggcgggcctcggggccaagatcgaactgtctgacacgaaggccgcttga

SEQ ID NO: 9 - DNA, 195 bp, synthetic construct
  source 1..195: mol_type = other DNA
gctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatc

SEQ ID NO: 10 - DNA, 210 bp, synthetic construct
  source 1..210: mol_type = other DNA
aagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcatttattgcaaagataaagcagattcctctagtacaagtggggaacaaaataacgtggaaaagagctgtcct

SEQ ID NO: 11 - DNA, 183 bp, synthetic construct
  source 1..183: mol_type = other DNA
gccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataa

SEQ ID NO: 12 - DNA, 217 bp, synthetic construct
  source 1..217: mol_type = other DNA
  modified_base 78: mod_base = i
  modified_base 114: mod_base = i
atcgttcaaacatttggcaataaagtttcttaagattgaatcctgttgccggtcttgcgatgattatcatattaattnctgttgaattacgttaagcatgtaataattaacatngtaatccatgacgttatttatgagatgggtttttatgattagagtcccgcaattatacatttaatacgcgatagaaaacaatatagcgcgcaaactaggataa

SEQ ID NO: 13 - DNA, 121 bp, synthetic construct
  source 1..121: mol_type = other DNA
gtttcgctcatgtgttgagcgtataagaaacccttagtatgtatttgtatttgtaaaatacttctatcaataaaatttctaattcctaaaaccaaaatccagtactaaaatccagatcccc

SEQ ID NO: 14 - DNA, 795 bp, synthetic construct
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga